(19) United States
(12) Patent Application Publication
(10) Pub. No.: US 2025/0201404 A1
(43) Pub. Date: Jun. 19, 2025

(54) RNA-BASED METHOD FOR SEIZURE ASSESSMENT

(71) Applicant: MOREHOUSE SCHOOL OF MEDICINE, Atlanta, GA (US)

(72) Inventor: Robert MELLER, Covington, GA (US)

(21) Appl. No.: 18/539,603

(22) Filed: Dec. 14, 2023

(51) Int. Cl. G16H 50/20; G16B 30/10; G16B 35/10; G16B 35/20; G16H 20/10

(52) U.S. Cl. CPC G16H 50/20; G16B 30/10; G16B 35/10; G16B 35/20; G16H 20/10

(57) ABSTRACT

A method to distinguish epileptic seizure from psychogenic non-epileptic seizure using blood RNA profiles. This assay involved the collection of a blood sample from a patient upon admission to a medical facility, the extraction of RNA from the blood sample, detection of RNA expression patterns and use of bioinformatic tools to detect a profile that matches that of psychogenic non-epileptic seizure vs other forms of seizure, or acute neurological crisis.

This application was made with government support under grant NS116762 awarded by the National Institutes for Health. The government has certain rights in the application.

### FIELD

The present disclosure relates generally to the field of neurology and, in particular, to methods for assessing and treating seizures.

### BACKGROUND

A seizure is a temporary loss of control. Seizures often, but not always, accompanied by convulsions, temporary loss of consciousness (TLOC), or both. Most common type of seizures are epileptic seizures, or seizures caused by sudden abnormal electrical discharges in the brain. Non-epileptic seizures and functional seizures (now termed psychogenic non-epileptic seizures (PNES), on the other hand, are not accompanied by abnormal electrical discharges. Non-epileptic seizures and functional seizures have no identifiable physical cause, but they are believed to be physical reactions to psychological stresses. Indeed, PNES is considered a dissociative condition in the latest DSM guide to psychological disorders.

The retrospective diagnosis of a seizure is challenging. A TLOC may be caused by multiple clinical conditions, and most seizures are unwitnessed by medical personnel, making their verification difficult. A clinical diagnosis of epilepsy following a seizure is typically based on clinical history, but the accuracy is limited. A seizure diagnosis can be confirmed using acute EEG during epilepsy monitoring, but this approach is time consuming and costly. Moreover, many patients with a seizures/epilepsy have normal EEGs between seizures. This diagnostic uncertainty results in delays to treatment, especially for PNES. Currently, there are no approved tests to retrospectively differentiate an epileptic seizure from other conditions that present as TLOC. Accordingly, there is a need for point-of-care diagnostic tools to assist in the accurate and timely diagnosis of epileptic seizure and to distinguish between patients with an epileptic seizure (approx. 40%) from those with non-epileptic seizures/PNES/functional seizures (10-20%), and those with non-seizure events such as syncope (25%) and physiological non-epileptic events (migraine etc.) (10%).

### SUMMARY

One aspect of the application relates to a method for retrospectively assessing a suspected seizure event in an individual and treating the individual, if necessary, according to the assessment. In some embodiments, the method comprises the steps of (1) generating a transcriptome profile of a seizure suspect from a blood sample obtained from the seizure suspect after a suspected seizure event, (2) comparing the transcriptome profile of the seizure suspect to reference transcriptome profiles of seizure patients; (3) determining whether the seizure suspect suffered a seizure based on a result of the comparing step and, (4) if the seizure suspect is deemed to have suffered a seizure in step (3), determining a cause of seizure. In some embodiments, the method further comprises the step of treating the seizure suspect with an appropriate seizure treatment based on the cause of seizure.

Another aspect of the present application relates to a method of enhancing treatment of epileptic seizures. The method comprises the steps of obtaining a whole blood sample from a subject, wherein the subject is suspected to have experienced an epileptic seizure, preparing an RNA library from the whole blood sample and sequencing the RNA library, determining differential expression of a plurality of RNA sequences comprised within the RNA library, wherein the plurality of RNA sequences comprises non-coding RNA (ncRNA), creating a transcriptome profile based on the differential expression of the RNA sequences, comparing the transcriptome profile to a reference transcriptome profile for epileptic seizure, determining whether the subject suffered an epileptic seizure, based on the result of the comparing step, and treating the subject with a therapy for epileptic seizure if the subject is deemed to have suffered an epileptic seizure in the determining step.

In some embodiments, the reference transcriptome profile for epileptic seizure is a reference transcriptome profile having the same sex and race characterization as the subject.

In some embodiments, the therapy comprises the step of administering to the subject, an effective amount of one or more drugs selected from the group consisting of brivaracetam, ezogabine, pregabalin, cannabidiol oral solution, felbamate, primidone, carbamazepine, fenfluramine, rufinamide, carbamazepine-XR, gabapentin, stiripentol, cenobamate, lacosamide, tiagabine hydrochloride, lamotrigine, clobazam, levetiracetam, topiramate, clonazepam, levetiracetam XR, topiramate XR, diazepam nasal, lorazepam, valproic acid, diazepam rectal, oxcarbazepine, vigabatrin, divalproex sodium-ER, phenobarbital, eslicarbazepine acetate, phenytoin and ethosuximide.

Another aspect of the present application relates to a method for treating epileptic seizures in a subject. The method comprises the steps of generating a transcriptome profile from a blood sample obtained from the subject, confirming that the subject suffered an epileptic seizure by identifying epileptic seizure RNA signatures in the transcriptome profile of the subject, wherein the presence of epileptic seizure RNA signatures indicates that the subject suffered an epileptic seizure, and treating the subject confirmed of epileptic seizure with a therapy for epileptic seizure.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1 shows an overview for using blood RNA profiles for retrospective seizure detection.

FIG. 2 shows analysis of 91 transcriptomes for quantification of reads to Ensembl-based annotation guide and custom annotation guide created from stringtie2 generated sample gtfs which are merged with the ensembl108 gft. Data were quantified using Partek Genomics Suite software to generate percentage of aligned reads to each genomic region. Of note only exonic regions are individually quantified by software. Data shown is mean =/−sd.

FIG. 3 shows that blood RNA profiles can identify the occurrence of an induced seizure. Panel A. Hierarchical cluster analysis of differentially expressed RNA in blood between control and seizure-treated rats (1-way ANOVA; FDR<0.05; ±2.0-fold change) (horizontal represents individual animals; vertical represents individual gene expression values). Panel B. Comparison of the blood RNA profiles of control animals and animals with an electrically evoked seizure. Principle component analysis (PCA) of the profiles show a clear delineation between blood RNA profiles generated from rats who had seizures compared to the controls.

FIG. 4 shows principal component analysis of differentially expressed exons in patients with seizure at 6 h. Panel A. PCA of EEG confirmed seizure (RED) vs EEG confirmed no seizure (blue). Image created by gene expression pattern of 66 genes (see list). Panel B. PCA of differentially expressed genes, contrasted by seizure type (ILAE definitions). PCA was performed using Partek, using gene expression values from 81 genes. Panel C. PCA analysis of DEGS from panels A and B combined, note the separation of PNES seizures from other seizure types.

FIG. 5 shows principal component analysis of differentially expressed genes in patients with seizure at 24 h. Panel A. PCA was performed using Partek, using 55 differentially expressed genes between seizure and non-seizure events. Panel B PCA was performed using Partek, using gene expression values from 59 genes, contrasted by seizure type (ILAE definitions). Panel C. PCA analysis of DEGS from panels A and B combined, note the separation of PNES seizures from other seizure types.

FIG. 6 shows that hierarchical cluster analysis and PCA reveal differential splicing following seizures, which distinguishes PNES from focal seizures and generalized seizures. Panel A. Hierarchical clustering of transcript usage. Note the lower sections shows the clustering of transcript expression patterns in PNES samples vs EEG confirmed samples. Panel B shows the PCA analysis of the same 88 differentially expressed transcript values shown in Panel A. Panel C shows PCA analysis of Alternative transcript expression That distinguishes different types of focal and generalized seizures. Panel D Shows principal component analysis also shows strong clustering of GTC, FTC and FIA samples away from respective baseline samples.

FIG. 7 shows that gene expression patterns change over time in patients with EEG-confirmed seizure and in patients with no EEG change seizure (PNES). Panel A: Time course of genes identified at 4-6 h time point as being differentially expression in patients with EEG change seizure. Panel B: Time course of genes identified at 24 h time point as being differentially expression in patients with EEG change seizure. Panel C: Time course of genes identified at 4-6 h time point as being differentially expression in patients with no EEG change seizure (PNES). Panel D: Time course of genes identified at 24 h time point as being differentially expression in patients with no EEG change seizure (PNES). Analysis reveals 187 genes show differential expression vs baseline control following a seizure. In contrast, 61 genes show differential expression in PNES patients at 4-6 h and 24 h (e.g., at discharge) post event compared to baseline. These data show some RNAs are temporally expressed following a seizure, and that temporal patterns of gene expression may enable the determination of when a seizure occurred in the past.

FIG. 8 is a PCA showing discriminant whole blood RNA profiles of patients who were originally designated as stroke mimics, but later adjudicated to have suffered seizure (red) from those patients who were non-seizure stroke mimics (blue).

FIG. 9 shows gender-specific gene expression profiles may be more accurate than a mixed sex model. Data from a previous study (Meller et al., Ann Clin Trans Neurol. 3:70-81, 2016) was refined, and only age and sex matched MCA stroke and controls used. Panel A. Hierarchical clusters of differentially expressed exon values in male only participants. Panel B. Hierarchical cluster of differentially expressed exon values in female only group. Panel C. Venn diagram showing overlap of exons in models identifying mixed gender, or sex specific changes following MCA. Note the lack of common genes in sex specific populations. Panel D. Power analysis modeling of sex specific data (female) to determine sample size (x axis) and power (y axis) to detect 2-fold changes with FDR 0.05 (modeled in SPAA).

FIG. 10 shows how correcting for sex and race reduces the number of DE genes identified in blood following seizures but may enhance accuracy. Data from 4-6 h samples were also subjected to differential expression analysis, with and without correction for race and gender. Genes showing significant 1.5-fold changes were subjected to Principal component analysis. Panel A shows sources of variation following linear modeling of the data using 4 contrast variables (race/gender/time of seizure 8 EEG change) with sample ID correction for random variables. Panel B shows sources of variation following 5 linear modeling of the data using 4 contrast variables. overlap of genes called FDR p<0.05 1.5 fold change. Panel C shows PCA results of corrected differentially expressed genes in yes vs no 4-6 h group (compare to FIG. 4, Panel A).

FIG. 11 shows receiver operating curve (ROC) analysis of calculated PC1 from FIG. 4, Panel A for ability to discriminate between EEG confirmed seizure patients vs. no seizure patients and baseline controls (AUC 1.0, at cutoff of −4.2, 100% specificity, 100% sensitivity).

FIG. 12 shows a workflow for using RNA expression values to generate models for diagnosis prediction.

While the present disclosure will now be described in detail, and it is done so in connection with the illustrative embodiments, it is not limited by the particular embodiments illustrated in the figures and the appended claims.

### DETAILED DESCRIPTION

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

### I. Definitions

As used herein, the following terms have the meanings ascribed to them unless specified otherwise.

The term “library” as used herein, refers to a collection of polynucleotides derived from nucleic acid sequences of a particular tissue, in particular RNA or cDNA. The polynucleotides of a library may be, but are not necessarily, cloned into a vector or set in a microarray.

The terms “nucleic acid” “polynucleotide” and “oligonucleotide” may be used interchangeably herein and refer to a deoxyribonucleotide or ribonucleotide polymer in either single- or double-stranded form. A “subsequence” or “segment” refers to a sequence of nucleotides that comprise a part of a longer sequence of nucleotides.

A “gene,” for the purposes of the present disclosure, includes a DNA region encoding a gene product. The region can also include DNA regions that regulate the production of the gene product, whether or not such regulatory sequences are adjacent to coding and/or transcribed sequences. This term in science also encompasses RNAs which are expressed by a cell, but that are not translated into a protein, such as a non-coding RNA, micro RNA, piRNA, etc. Accordingly, a gene can include, without limitation, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA, or a novel RNA whose function is as yet to be determined) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

The term “transcriptome profile” as used herein, refers to an RNA expression profile obtained from sequencing RNAs extracted from a blood sample. The extracted RNAs may contain both coding and non-coding RNAs and may be pre-treated to enrich or deplete certain types of RNAs prior to sequencing.

The term “whole transcriptome profile” as used herein, refers to an RNA expression profile obtained from sequencing total RNAs extracted from a blood sample, without enrichment or depletion of any types of RNAs prior to RNA-sequencing.

The term “non-coding RNA” (ncRNA) refers to an RNA molecule that is not translated into a protein. The number of non-coding RNAs within the human genome is unknown; however, recent transcriptomic and bioinformatics studies suggest that there are thousands of them. Many of the newly identified ncRNAs have not been validated for their function. It is also likely that many ncRNAs are non-functional (sometimes referred to as junk RNA), and are the product of spurious transcription. Abundant and functionally important types of non-coding RNAs include transfer RNAs (tRNAs) and ribosomal RNAs (rRNAs), as well as small RNAs such as microRNAs, siRNAs, piRNAs, snoRNAs, snRNAs, exRNAs, scaRNAs and the long ncRNAs such as Xist and HOTAIR. The ncRNA may have some associated activity that may be deleterious. Most often the major concern is whether it will be translated into short random peptides.

### II. Method of Retrospective Diagnosing Seizure and Treating Seizure

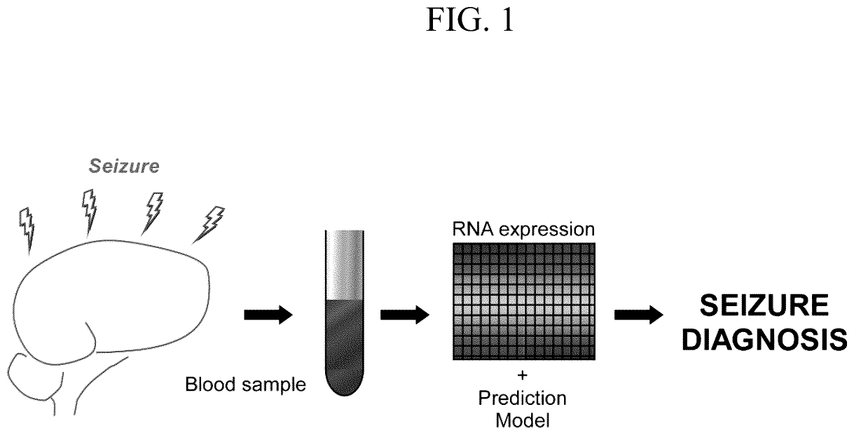

One aspect of the present application relates to a method for retrospectively diagnosing seizure. As shown in FIG. 1, Blood sample is obtained from a seizure suspect. The RNA expression profile of the blood sample is generated by RNA sequencing and analyzed in a prediction model to generate a seizure assessment.

The method can be used to differentiate epileptic seizure from psychogenic non-epileptic seizure. In some embodiments, the method comprises the steps of generating a transcriptome profile of a seizure suspect from a blood sample obtained from the seizure suspect after a suspected seizure event, comparing the transcriptome profile of the seizure suspect to reference transcriptome profiles of seizure patients, and determining whether the seizure suspect suffered seizure based on a result of the comparing step.

In some embodiments, the method further comprises the steps of extracting RNA from the blood sample of the seizure suspect and generating a transcriptome profile based on the RNA extracted from the blood sample. In some embodiments, the method further comprises the step of treating the seizure suspect with a seizure treatment if the seizure suspect is determined to have suffered a seizure.

### Seizure Suspect

A seizure suspect is a subject who is suspected of experienced a seizure. In some embodiments, the seizure suspect is a mammal. In some embodiments, the seizure suspect is a human. In some embodiments, the seizure suspect exhibits one or more symptoms of seizure. Examples of seizure symptoms include, but are not limited to, staring, jerking movements of the arms and legs, stiffening of the body, loss of consciousness, breathing problems or stopping breathing, loss of bowel or bladder control, falling suddenly for no apparent reason, especially when associated with loss of consciousness, not responding to noise or words for brief periods, appearing confused or in a haze, nodding head rhythmically, when associated with loss of awareness or loss of consciousness, and periods of rapid eye blinking and staring.

While exhibiting seizure symptoms, the seizure suspect may not suffered a seizure (such seizure suspects are also referred to as “seizure mimics” in this application.

In some embodiments, the seizure suspect exhibited symptoms of, or is suspected of having, an epilepsy seizure. In some embodiments, the seizure suspect exhibited symptoms of, or is suspected of having, a non-epilepsy seizure.

In some embodiments, the seizure suspect is asymptomatic but has a risk or predisposition to experiencing seizure, e.g., based on genetics, a related disease condition, environment or lifestyle.

### Sample Collection And RNA Extraction

The blood sample of the seizure suspect should be collected within 48 hours of the suspected seizure event. In some embodiments, the blood sample is collected upon admission at a medical facility. In some embodiments, the blood example is collected within 6, 8, 12, 24 or 48 hours post a suspected seizure. In some embodiments, the blood sample is collected 4-6 hours post a suspected seizure. In some embodiments, the blood sample is collected 4-12 hours post a suspected seizure. In some embodiments, the blood sample is collected 4-24 hours post a suspected seizure. In some embodiments, the blood sample is collected 4-48 hours post a suspect seizure.

In some embodiments, the blood is collected with the PAXgene blood collection tubes. A whole blood sample contains six types of cells: red blood cells, neutrophils, eosinophils, basophils, lymphocytes, and monocytes, as well as platelets.

RNA is extracted from the blood sample using methods well known in the art. Briefly, the blood sample is treated with reagents that lyse blood cells and inactivate cellular RNases. Cellular RNA is then isolated and subjected to further analysis. Examples of RNA extraction kits include, but are not limited to, Tempus blood RNA isolation system (ThermoFisher Scientific), RiboPure-Blood kit (ThermoFisher Scientific), LeukoLOCK (ThermoFisher Scientific), and QiaCube (Qiagen). In some embodiments, the RNA is extracted with QiaCube. In some embodiments, the RNA is from a whole blood sample. In some embodiments, one or more specific cell components of the blood, such as red blood cells, neutrophils, eosinophils, basophils, lymphocytes, and/or monocytes, may be isolated or purified, prior to RNA extraction, or determined by single cell sequencing methodologies.

### Generation Of Transcriptome Profile

RNA extracted from the blood sample is subjected to RNA-sequencing (also referred to as RNA-Seq) to generate a transcriptome profile of the seizure suspect. RNA-Seq uses high-throughput sequencing to illuminate the existence and relative quantities of RNA molecules at a given moment in a biological sample. In addition to mRNA transcripts, RNA-Seq can also look at different populations of RNA (such as miRNA or tRNA) in the RNA extract.

RNA-Seq works in concert with a range of high-throughput DNA sequencing technologies. However, prior to sequencing of the extracted RNA transcripts, several key processing steps are performed. Methods differ in the use of transcript enrichment, fragmentation, amplification, single or paired-end sequencing, and whether to preserve strand information. One of ordinary skill will understand that the particular type or form of RNA-Seq is not limiting on the application discussed herein.

In the case of blood, the RNA extract may contain a large amount of ribosomal RNA (rRNA) and non-coding RNA (ncRNA). The sensitivity of any given RNA-Seq analysis can be enhanced by enriching RNA classes of interest, while depleting known abundant RNAs. If so desired, the mRNA molecules can be removed by using oligonucleotides probes that bind their poly-A tails or enriched by using primers with polyT sequences. Alternatively, abundant but uninformative ribosomal RNAs (rRNAs) can be removed by ribo-depletion by hybridization to probes designed to target specific rRNA sequences (A). However, ribo-depletion may also introduce some bias via non-specific depletion of off-target transcripts. Gel electrophoresis and extraction can be used to purify small RNAs, such as micro RNAs, by their size.

In a preferred embodiment, the RNA extract is subjected to RNA-seq without removal of rRNA or ncRNA, and without enrichment of mRNA. RNA-seq of such RNA extraction permits the identification of both coding and non-coding RNAs (whole transcriptome). Whole transcriptome analysis detects changes in exon expression and alternative transcript splicing events that occur rapidly following seizure, thus allowing more accurate biomarker panel profiling/RNA signature determination for seizure and seizure subtype differentiation.

In some embodiments, rRNA is removed from the RNA extract prior to RNA-Seq. In some embodiments, rRNA is not removed from the RNA extract prior to RNA-Seq. In some embodiments, rRNA is not removed from the RNA extract prior to RNA-Seq but IRNA sequences are bioinformatically removed following RNA-seq.

In some embodiments, ncRNA is removed from the RNA extract prior to RNA-seq. In some embodiments, ncRNA is not removed from the RNA extract prior to RNA-seq. In some embodiments, ncRNA is not removed from the RNA extract prior to RNA-Seq but ncRNA sequences are bioinformatically removed following RNA-seq.

In some embodiments, the RNA extract is enriched for mRNA prior to RNA-Seq. In some embodiments, the RNA extract is not enriched for mRNA prior to RNA-Seq.

In some embodiments, the RNA extract is subjected to RNA-seq without removal of rRNA or ncRNA, and without enrichment of mRNA. RNA-seq of such RNA extraction permits the identification of both coding and non-coding RNAs (whole transcriptome). Whole transcriptome analysis detects changes in exon expression and alternative transcript splicing events that occur rapidly following seizure, thus allowing more accurate biomarker panel profiling/RNA signature determination for seizure and seizure subtype differentiation. In some embodiments, NCRNA and/or rRNA sequences are bioinformatically removed following RNA-seq

In some embodiments, the extracted RNA is fragmented prior to RNA-Seq. Fragmentation may be achieved by chemical hydrolysis, e.g bulization, sonication, or reverse transcription with chain-terminating nucleotides. Alternatively, fragmentation and cDNA tagging may be done simultaneously by using transposase enzymes. One of ordinary skill will understand that the particular method of preparing a transcriptome for sequencing is not limiting on the application discussed herein.

The extracted RNA can be sequenced in just one direction (single-end) or both directions (paired-end). A single-end sequence is usually quicker to produce, cheaper than paired-end sequencing and sufficient for quantification of gene expression levels. Paired-end sequencing produces more robust alignments/assemblies, which is beneficial for gene annotation and transcript isoform discovery. Strand-specific RNA-Seq, methods preserve the strand information of a sequenced transcript. Without strand information, reads can be aligned to a gene locus but do not inform in which direction the gene is transcribed. Stranded-RNA-Seq is useful for deciphering transcription for genes that overlap in different directions and to make more robust gene predictions in non-model organisms. One of ordinary skill will understand that the particular strands used in sequencing are not limiting on the application described herein.

The RNA-Seq may be performed using methods well known in the art. Examples of such methods include quantitative polymerase chain reaction (qPCR), high throughput multiplex nucleic acid sequencing and nanopore sequencing. In some embodiments, the RNA-Seq is perform with the Ion Torrent Platform (ThermoFisher). In some embodiments, the RNA extract is not primed with a polyT primer, thus reducing bias for polyA and 3′ transcripts. In some embodiments, the RNA-Seq libraries are stranded libraries.

### Transcriptome Assembly

The raw data generated by sequencing is then processed to generate a transcriptome profile for the seizure suspect. Transcriptomics methods are highly parallel and require significant computation to produce meaningful data for both microarray and RNA-Seq experiments. RNA-Seq analysis generates a large volume of raw sequence reads which have to be processed to yield useful information. Data analysis usually requires a combination of bioinformatics software tools that vary according to the experimental design and goals. The process can be broken down into four stages: quality control, alignment, quantification, and differential expression. Most popular RNA-Seq programs are run from a command-line interface, either in a Unix environment or within the R/Bioconductor statistical environment.

Sequence reads are not perfect, so the accuracy of each base in the sequence needs to be estimated for downstream analyses. Raw data is examined to ensure: quality scores for base calls are high, the GC content matches the expected distribution, short sequence motifs (k-mers) are not over-represented, and the read duplication rate is acceptably low. Several software options exist for sequence quality analysis, including FastQC and FaQCs. Abnormalities may be removed (trimming) or tagged for special treatment during later processes.

In order to link sequence read abundance to the expression of a particular RNA, transcript sequences are aligned to a reference genome. The key challenges for alignment software include sufficient speed to permit billions of short sequences to be aligned in a meaningful timeframe, flexibility to recognize and deal with intron splicing of eukaryotic mRNA, and correct assignment of reads that map to multiple locations. Software advances have greatly addressed these issues and increases in sequencing read length reduce the chance of ambiguous read alignments. One of ordinary skill will understand the choice of high-throughput sequence aligners that are available and may be selected for analyses.

Alignment of primary transcript mRNA sequences derived from eukaryotes to a reference genome requires specialized handling of intron sequences, which are absent from mature mRNA. Short read aligners perform an additional round of alignments specifically designed to identify splice junctions, informed by canonical splice site sequences and known intron splice site information. Identification of intron splice junctions prevents reads from being misaligned across splice junctions or erroneously discarded, allowing more reads to be aligned to the reference genome and improving the accuracy of gene expression estimates. Since gene regulation may occur at the mRNA isoform level, splice-aware alignments also permit detection of isoform abundance changes that would otherwise be lost in a bulked analysis.

In some embodiments, RNA sequencing data (reads) are aligned to the human genome (Grch38) using a custom script combining STAR and Bowtie2 alignment software. In some embodiments the data are aligned or mapped with alternative software (e.g. minimap2, hisat, bwa, kraken, salmon etc). in some embodiments the data are aligned to complete end to end versions of the human genome (such as Chm-13 from the Telomere to telomere project), or ancestry specific reference genomes, or a human pan-genome model. Gene aligned reads (Counts data) are analyzed using Partek Genomics Studio. All data are normalized to reads per kilobase per million aligned reads, and all data are further normalized by dividing by the trimmed mean of the rkpm values. Gene expression and transcript usage is determined using linear models following correction for batch (in linear model). In some embodiments alternative normalization methods are utilized including counts, counts per million or transcript per million aligned reads approaches.

In some embodiments, custom annotation guides are created to quantify novel RNAs that align to previously unannotated regions of human genome. Briefly, a custom annotation guide is created using software to first identify the genomic origin of a detected RNA in the sequencing data. This creates a datafile (.gtf or .gff) of genomic regions, which are compiled for all samples, and then compared and merged with the published available reference annotation guide using various software. This new annotation guide will contain all genomic regions that express RNA, which are then used by current RNA counting tools to derive the RNA expression.

Quantification of sequence alignments may be performed at the gene, exon, or transcript level. Typical outputs include a table of read counts for each feature supplied to the software; for example, for genes in a general feature format file. Gene and exon read counts may be calculated quite easily using HTSeq, for example. Quantitation at the transcript level is more complicated and requires probabilistic methods to estimate transcript isoform abundance from short read information; for example, using cufflinks software. Reads that align equally well to multiple locations must be identified and either removed, aligned to one of the possible locations, or aligned to the most probable location. In some embodiments, alternative read counting software may be utilized, such as string-tie2.

Some quantification methods can circumvent the need for an exact alignment of a read to a reference sequence altogether. The kallisto software method combines pseudoalignment and quantification into a single step that runs two orders of magnitude faster than contemporary methods such as those used by tophat/cufflinks software, with less computational burden.

Once quantitative counts of each transcript are available, differential gene expression is measured by normalizing, modelling, and statistically analyzing the data. Most tools will read a table of genes and read counts as their input, but some programs, such as cuffdiff, will accept binary alignment map format read alignments as input. The final outputs of these analyses are gene lists with associated pair-wise tests for differential expression between treatments and the probability estimates of those differences.

### Determination of Seizure Cause Based on the Transcriptome Profile of the Seizure Suspect

The transcriptome profile of the seizure suspect is then compared to a database of seizure patient transcriptome profiles using mathematical clustering to identify patterns of gene expression and transcriptomic signature that are unique for each seizure subtype, patient type and/or treatment type. The database may further contain the transcriptome profiles of seizure mimics (individuals showing seizure symptoms but did not experience a seizure) transcriptome profiles. The seizure patient transcriptome profiles include, but are not limited to, general seizure transcriptome profiles that contain robust differentially expressed genes (DEGs) characteristic in all seizure patients, seizure subtype specific transcriptome profiles, such as epileptic seizure specific transcriptome profiles and non-epileptic seizure specific transcriptome profiles. The epileptic seizure specific transcriptome profiles may further include syncope-specific transcriptome profiles, TIA-specific transcriptome profiles, focal to bilateral tonic clonic (FTC) seizures-specific transcriptome profiles and with generalized tonic clonic (GTC) seizure-specific transcriptome profiles. In some embodiments, the transcriptome profiles are further divided into sub-transcriptome profiles characterized by gender, race, age, etc. to provide more accurate assessment of the transcriptome profile of the seizure suspect, and to distinguish epileptic seizures from psychogenic non-epileptic seizure, or psychogenic non-epileptic seizure from other forms of seizure, or acute neurological crisis.

As shown in the Examples of this application, individuals who suffered a seizure have unique RNA expression patterns (also referred to as RNA signatures) that are different from individuals who have not suffered a seizure. In addition, individuals who suffered different types of seizure showed different RNA expression patterns, thus allowing differentiation of seizure subtype based on the RNA expression patterns.

In some embodiments, the database further contains the biographical information, such age, sex, race, and marital status, as well as personal and family medical history of each individual. Such parameters may also be included in the algorithm for determining seizure subtype. In some embodiments, the database further contains blood transcriptome patterns of seizure patients who received seizure treatment. Such information may be used to evaluate treatment efficacy and safety of the treatment and may also serve as a predictor of therapeutic responses.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20, 25, 30, 35, 40, 45, 50, 55, 60 or more of the genes selected from the group consisting of TMEM179B, ID2, DHX8, ENSG00000218426, PTCD2, RPL10P16, THOC5, TYW5, ENSG00000232626, RPLPOP6, ITM2A, F2R, C11orf58, PACS1, JUND, ENSG00000283907, ERH, MBD3, SLC25A30, PRDX3, YWHAZP3, RPL4P4, YBX1P10, HDAC9, GPX4, MESD, AHCTF1P1, RPL13AP5, RABIF, ARL6IP5, ENSG00000218175, KDM2B, SNX17, GPX1, EIF2S3B, ENSG00000270066, LANCL1, PTGER4, ANXA7, MED29, PTGER2, EEF1A1P11, SP1, MFHAS1, ENSG00000244313, GSTP1, EEF1A1P4, TRBC1, PARP6, PCID2, PIGV, FAN1, INPP5F, UTP23, FECH, FBXO9, RPS2P46, ANKRD10, HPCAL1, SLC35E1, LY75-CD302, DUSP7, PSMG2, TRIM24, MRS2 and KLF2. These RNA signatures allow the determination of the likelihood of the occurrence of a seizure event in a seizure suspect.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20, 25, 30, 35, 40, 45, 50, 55, 60, 65, 70, 75, 80, 85 or more of the gene transcripts selected from the group consisting of ENST00000397797>HBA1, ENST00000510187>SQSTM1, ENST00000695862>CDC42, ENST00000373298>ITM2A, ENST00000652583>ZFP36, ENST00000530945>CFL1, ENST00000417053>ENSG00000232626, ENST00000507943>BODIL1, ENST00000693666>GOLGA3, ENST00000503742>UBE2D3, ENST00000553630>CALM1, ENST00000544690>TYROBP, ENST00000250896>MKNK2, ENST00000427401>ZNF737, ENST00000273223>RPL32, ENST00000228136>C11orf58, ENST00000415292>YWHAZP3, ENST00000394878>RPLPOP6, ENST00000492974>ENSG00000218426, ENST00000695948>TRERF1, ENST00000319211>F2R, ENST00000325000>RPL10P16, ENST00000444752>YBX1P10, ENST00000393238>TMCC1, ENST00000273258>ARL6IP5, ENST00000422486>RPL4P4, ENST00000602755>ENSG00000270066, ENST00000367262>RABIF, ENST00000638356>ENSG00000283907, ENST00000202677>RALGAPA2, ENST00000420969>AHCTF1P1, ENST00000394432>RASGRP2, ENST00000333219>ING1, ENST00000439189>RPL13AP5, ENST00000398606>GSTP1, ENST00000304414>ARL6IP1, ENST00000299927>ZNF592, ENST00000372919>ANXA7, ENST00000276282>MFHAS1, ENST00000405359>ENSG00000218175, ENST00000380680>UBL3, ENST00000421246>CDC42P6, ENST00000467930>ENSG00000244313, ENST00000252818>JUND, ENST00000376802>HLA-A, ENST00000245457>PTGER2, ENST00000162749>TNFRSF1A, ENST00000415278>EEF1A1P11, ENST00000298510>PRDX3, ENST00000457354>TMX1, ENST00000508832>MALAT1, ENST00000359165>EEF1A1P4, ENST00000607003>SAP18, ENST00000633705>TRBC1, ENST00000349213>ARID4B, ENST00000490163>UBXN4, ENST00000554465>SRSF5, ENST00000234310>PPP3R1, ENST00000484897>RPSAP58, ENST00000458332>RPS2P46, ENST00000511164>RAC1P2, ENST00000377474>KCTD12, ENST00000382873>FECH, ENST00000676189>ACTB, ENST00000683488>ATM, ENST00000307808>AFF1, ENST00000498502>MBNL1, ENST00000615892>PPP1R18, ENST00000440859>CBLL1, ENST00000673846>BIRC3, ENST00000392563>GRB2, ENST00000670229>FMNL1-DT, ENST00000462885>RPL18AP3, ENST00000650601>HBD, ENST00000363046>RMRP, ENST00000377795>CD74, ENST00000498661>ORAI2, ENST00000392754>FAM53B, ENST00000248071>KLF2, ENST00000635620>DENND4A, ENST00000335007>PPP1CC, and ENST00000274031>SETD7. These RNA signatures allow the determination of the likelihood of the occurrence of a seizure event in a seizure suspect.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20, 25, 30, 35, 40, 45, 50 or more of the genes and/or gene transcripts selected from the group consisting of ADAMTSL4, ADH5, AQP9, BCL2A1, BCL6, BDH1, BIRC3, BTNL8, Clorf52, CAMK4, CCNJL, CD200R1, CEACAMI, CEBPB, CEP290, CHSY1, CLEC4D, CLEC4E, CR1, CRISPLD2, DCAF13, DHRS13, DPP4, EEF1A1P22, ENSG00000218426, ENSG00000238035, ENSG00000241860, ENSG00000259600, ENSG00000274272, ENSG00000288932, ENSG00000289013, ENSG00000290021, ENSG00000290385, ENSG00000290585, ENSG00000290937, ENSG00000291215, ENSG00000291221, F5, FAM157C, FAM157D, FCAR, FKBP5, FKTN, FOSL2, FUT7, GDPD5, GRAP, GSEC, GTF2IRD2B, HSPA6, ILIR1, IRAG1, KCNE1, KCNJ15, KPNA5, LEO1, LIMK2, LINC01001, LINC01002, LIPN, LMNB1, LYRM7, MANIC1, MMP25, MMP9, MTCYBP23, MTND5P32, MXD3, NAIP, PCDH9, PELATON, PFKFB3, PFKFB4, PGS1, PHC2, PLEKHA8, PLIN5, PPA1, QPCT, RALGAPA1P1, RNF157, RPL13AP25, RPL13AP5, RPL3P4, RPL4P4, RPS23P8, RPS26, RRP1B, S100A8, S100P, SCARF1, SFXN1, SLAMF1, SLC12A9, SLC37A3, SLC39A10, SNORD14D, TBKBP1, THEMIS, TMEM263, TMX2, TOMM7, TP53111, TRBC1, TXNDC15, TYW1, UBIAD1, VPS9D1, WDFY3, ZBTB5, ZC3H12D, ZNF260, ZNF480, ZNF568, ZNF7, ZSCAN12, ACP6, ADM, AK3, ALG2, ALG5, ANK3, ATP1B3, B4GALT5, BASP1, CCDC141, CD8B, CLEC4A, CUTA, DBP, DMAP1, ECI2, ENSG00000249624, ENSG00000291066, GK, HAUS4, IFT80, KLHDC4, KREMEN1, LILRA5, LINC01550, LSG1, MRTFB, NAMPTP1, NDUFA12, NLRC4, OSCAR, PGLYRP1, PLSCR1, POLA1, RYK, SELENOW, SIGLEC5, SMG1P1, ST20, TESPA1, TMEM18, TNFRSF25, WDR27, ZNF441, ZNF559, ZNF594, ACSL1, ADGRG3, AKRIB1, ALPL, ANXA3, BCL3, CNTNAP3C, CYSTM1, DYSF, ENSG00000290890, GNG10, GOLT1B, HRH2, LINC01127, LRG1, MGAM, MGAM2, MTARC1, PROK2, S100A12, SIPA1L2, SLC11A1, TGFA, TLR5, and TRPM2. These RNA signatures allow the determination of the likelihood of the occurrence of a seizure event in a seizure suspect.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20, 25, 30, 35, 40, 45, 50 or more of the genes and/or gene transcripts selected from the group consisting of TTLL3, GPX1, GTF3C1, CYTOR, RNASEL, SEPHS2, FECH, PACS1, RAB5IF, DUSP7, MYNN, FBXO9, TRIM24, DHX8, F2R, SNX17, ERF, HNRNPKP4, GTF2IRD2, POGLUT1, DUT, TMEM30A, CBLL1, ENSG00000291117, ADAM9, TSNAX, PGAP6, EEF1A1P11, ZC3H10, ERH, PTGER2, CCP110, DENND6A, COA1, PTGER4, MAN2A2, RALGPS1, TMEM179B, CDC42P6, LINC01355, MFN2, TYW5, PSMG2, H3P47, ZNF761, RPL10P16, EIF2S3B, SMARCC1, KLF2, MFSD8, EXOSC2, RABIF, PRDX3, GGPS1, SAP18, MBD3, AHCTF1P1, YWHAZP3, ENSG00000270066, MOGS, CARD8-AS1, KIAA2013, HPCAL1, SELENON, CDC42BPB, PCID2, TGFBRAP1, ENSG00000244313, THUMPD3, RAC1P2, ENSG00000283907, VDAC3, TBC1D25, PRRC2C, SLC16A5, ANKRA2, PTCD2, and TRAM1. These RNA signatures allow the determination of the likelihood of the occurrence of a seizure event in a seizure suspect.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20, 25, 30, 35, 40, 45, 50 or more of the genes selected from the group consisting of RN7SKP176, RN7SKP255, PTGER4, RN7SL396P, TAOK1, DEAF1, YBX1P10, DAP, RN7SKP203, TVP23C, ZNF687, UBBP4, ZBTB7A, PGLS, ORAII, HBZP1, DCTN2, AGPAT1, FAM172A, PRELID3B, LLGL2, RNVU1-31, D2HGDH, IQCN, ACTBP2, RN7SL674P, PTPA, ZNF189, DPEP2, SNHG32, RN7SKP71, EDEM3, TSC22D2, SMURF1, ENSG00000283907, MAPIS, USP34, MTHFS, HBA1, RN7SL3, SF3B5, EPC1, MAN2C1, PCBP2P2, HBA2, MRTFB, SEC14LIP1, HK1, CALM3, RPS2P46, RABGEF1, AHCYL1, PSMC1, TECPR1 and YBX1P1. These RNA signatures allow the determination of the likelihood of the occurrence of a seizure event in a seizure suspect.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20 or more of the gene transcripts selected from the group consisting of ENST00000478004>PSMF1, ENST00000363673>RN7SKP176, ENST00000487791>HBA1, ENST00000484216>HBA2, ENST00000363442>RN7SKP255, ENST00000471086>RN7SL396P, ENST00000248437>TUBA4A, ENST00000472694>HBA1, ENST00000482565>HBA2, ENST00000230895>DAP, ENST00000444752>YBX1P10, ENST00000363618>RN7SKP203, ENST00000379419>PDE7A, ENST00000356444>SEMA4D, ENST00000354915>HBZP1, ENST00000598034>GMFG, ENST00000487283>ZAP70, ENST00000322357>ZBTB7A, ENST00000462494>ACTB, ENST00000605806>RNVU1-31, and ENST00000409064>KDM3A. These RNA signatures allow the determination of the likelihood of the occurrence of a seizure event in a seizure suspect.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20, 25, 30, 35, 40, 45, 50, 55 or more of the genes and/or gene transcripts selected from the group consisting of ENSG00000286813, FBX09, AHCYL1, ALOX12-AS1, ARHGAP19, ARID3B, ATP5MC2, C4orf3, C6orf47, CCDC28A, CHPF2, CTPS1, D2HGDH, DCTN2, DPEP2, DPH3, DST, EIF4ENIF1, ENSG00000289474, ESS2, FAM20B, FAM98A, GAS5, GDE1, GTPBP6, HERC6, IQCN, LIMK1, MTERF4, NELFE, NSUN3, PGLS, PPP2CA, PSMC1, RABGGTB, RANBP9, RBM18, RN7SKP255, RNVU1-31, SLC19A1, SMC3, SNHG29, SNHG32, SNORD100, SNORD30, SNORD34, SNORD41, SNORD5, SNORD69, SNORD91B, STRN, STX17, TDP2, TECPR1, TOP3A, TTF1, TVP23C, USP16, USP33, WDR6, and ZNF766. These RNA signatures allow the determination of the likelihood of the occurrence of a seizure event in a seizure suspect.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20, 25, 30, 35, 40, 45, 50, 55, 60, 65, 70, 75, 80 or more of the genes selected from the group consisting of BORCS8, CAD, DERL1, ENSG00000291067, MT-CO3, TMEM219, ABCA7, AKT3, ALPL, APMAP, ARL11, ARRB2, ASB1, CTNNA1, CYB5R4, DHX34, DNAJB6, DOCK5, EEF1G, ELAC2, ENSG00000290791, FAM8A1, FCHO2, FPR2, GCA, GLTID1, GMPR2, HEBP2, HYCC2, IL10RB, JAML, KCNJ15, KREMEN1, LAT2, LBH, LINC01127, LYAR, MPZL3, MSL1, NADK, NELFCD, NPEPPSP1, PELI1, PLXNC1, PPP2R5A, PSMC4, RNF149, RPL13A, RPL37A, SDHAF2, SIGLEC9, SLC11A1, SLC16A3, SLC9A8, SNORD110, SNX10, SOS2, TMEM120A, TMEM71, TMLHE, TNFRSF10C, TRBC2, TREML2, TSEN34, VNN3P, WRN, ZBTB38, ZNF516, CWF19L1, EIFIAD, GNB4, HNRNPA1P10, NSRP1, RPL13AP5, RPL6P27, RPL7P1, SP140L, TIGIT, DHPS, MBTPS1, and PPIE. These RNA signatures allow the determination of the type of seizure event occurred in a seizure suspect.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20, 25, 30, 35, 40, 45, 50, 55, 60, 65, 70, 75, 80, 85 or more of the gene transcripts selected from the group consisting of ENST00000330720>KDELR1, ENST00000361624>MT-CO1, ENST00000362079>MT-CO3, ENST00000519638>ERLIN2, ENST00000581241>CSNKID, ENST00000694951>IL17RA, ENST00000216146>RPL3, ENST00000217456>APMAP, ENST00000233143>TMSB10, ENST00000250360>SIGLEC9, ENST00000265085>CPEB4, ENST00000272519>RALB, ENST00000290200>IL10RB, ENST00000295317>RNF149, ENST00000326648>ZNF609, ENST00000329251>EEF1G, ENST00000339569>MSL1, ENST00000356864>TNFRSF10C, ENST00000367075>EZR, ENST00000368885>AMD1, ENST00000369535>NRAS, ENST00000374391>ALOX5, ENST00000374840>ALPL, ENST00000389120>RNF20, ENST00000391857>RPL13A, ENST00000395323>LBH, ENST00000395762>IL4R, ENST00000396946>CARD11, ENST00000397708>MCM3AP, ENST00000418596>HYCC2, ENST00000443185>ZNF516, ENST00000466254>TRBC2, ENST00000483722>TREML2, ENST00000487445>GCA, ENST00000492413>SLC11A1, ENST00000513001>ACSL1, ENST00000520106>SLA, ENST00000547026>ZNF641, ENST00000550811>TUBA1A, ENST00000561488>XPO6, ENST00000607197>HEBP2, ENST00000608318>ENSG00000290791, ENST00000613060>SLC6A6, ENST00000624694>HRH2, ENST00000627215>LINC01127, ENST00000672722>ARHGAP35, ENST00000673794>VNN3P, ENST00000678703>EEF1A1, ENST00000703699>GNB1, ENST00000706367>ACSL1, ENST00000398253>ZFP90, ENST00000439151>NSD1, ENST00000444945>HNRNPA1P10, ENST00000468161>RPL7P1, ENST00000526668>RPL8, ENST00000552951>NOPCHAP1, ENST00000683488>ATM, ENST00000683697>MYCBP2, ENST00000696270>TNRC6C, ENST00000646122>DDX3X, ENST00000360902>TRRAP, ENST00000367536>NCF2, ENST00000426496>PRRC2C, and ENST00000456586>RPL37A. These RNA signatures allow the determination of the type of seizure event occurred in a seizure suspect.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20, 25, 30, 35, 40, 45, 50, 55 or more of the genes selected from the group consisting of RBM7, ATP11B, DNAJC3, FAM91A1, FAR1, FBXL5, FOXN2, GCA, GLIPR2, HSDL2, HYCC2, ITM2B, LMBRD1, LPGAT1, MANSC1, MTMR6, NABP1, PELI1, PIP4P2, PSTPIP1, PTEN, RAB10, RAB5IF, RGL2, RNF149, RRM2B, SNHG1, SNHG32, SNORA31, SNORA81, SNORD11, SNORD110, SNORD25, SNORD27, SNORD30, SNORD35B, SNORD42B, SNORD50B, SNORD52, SNORD56, SNORD58B, SNORD6, SNORD60, SNX10, SPOPL, SULT1B1, TLR1, TMLHE, UBE2A, UBE2D1, WIPI1, ACTR8, CDC123, GANAB, PCYOX1, RPL36A, ZNF827, CNEPIR1 and MYCBP. These RNA signatures allow the determination of the type of seizure event occurred in a seizure suspect.

In some embodiments, the seizure patient transcriptome profiles contain RNA expression patterns/RNA signature of 1, 2, 3, 4, 5, 6, 7, 8, 9, 10, 11, 12, 13, 14, 15, 16, 17, 18, 19 20, 25, 30, 35, 40, 45, 50 or more of the gene transcripts selected from the group consisting of ENST00000336332>ZXDC, ENST00000338272>TMEM167B, ENST00000377461>CARD8, ENST00000492741>PSPC1, ENST00000233612>GCA, ENST00000251810>RRM2B, ENST00000260130>SDCBP, ENST00000280098>SPOPL, ENST00000295317>RNF149, ENST00000332180>WASHC4, ENST00000334398>TMLHE, ENST00000335181>PKM, ENST00000355356>MIER1, ENST00000358912>PELI1, ENST00000362607>SNORA31, ENST00000363981>SNORD27, ENST00000364884>SNORD52, ENST00000364995>SNORD50B, ENST00000365607>SNORD25, ENST00000366997>LPGAT1, ENST00000369856>FLNA, ENST00000374832>ALPL, ENST00000384693>SNORD30, ENST00000395788>SNHG32, ENST00000413522>SNORD56, ENST00000420627>FLNA, ENST00000432261>RNF24, ENST00000437150>GCA, ENST00000443799>GAS5, ENST00000458893>SNORD42B, ENST00000466047>MCMBP, ENST00000487445>GCA, ENST00000527983>HSPA8, ENST00000538654>SNHG1, ENST00000550811>TUBA1A, ENST00000620947>MCL1, ENST00000649266>ITM2B, ENST00000650315>OGFRL1, ENST00000688158>PTEN, ENST00000702934>GAS5, ENST00000339569>MSL1, ENST00000313367>OSBPL3, ENST00000357164>GM2A, ENST00000368003>UFC1, ENST00000376406>MDC1, ENST00000613780>MIAT, ENST00000673822>DDHD1, ENST00000677965>EIF3E, ENST00000700035>ARHGAP35, ENST00000703700>GNB1, and ENST00000480503>MYO1G. These RNA signatures allow the determination of the type of seizure event occurred in a seizure suspect.

In some embodiments, the database of seizure patient transcriptome profiles contains a database on predictive transcriptomic signatures that may serve as predictors of treatment outcome. The database of seizure patient transcriptome profiles may be updated from time to time with new data using artificial-intelligence and machine learning tools to improve accuracy of the diagnosis.

Comparison of the transcriptome profile of the seizure suspect to the seizure RNA profiles allow for the determination of (1) whether the seizure suspect suffered a seizure, and (2) if the seizure suspect suffered a seizure, the subtype of the seizure. In a particular embodiment, the comparison of the transcriptome profile of the seizure suspect to the seizure patients transcriptome profiles allows for the differentiation of ischemic seizure from hemorrhagic seizure.

In some embodiments, the seizure suspect is deemed to have suffered a seizure if the transcriptome profile of the seizure suspect matches to a reference transcriptome profile of seizure patients. A match is made when the profile is compared to a prediction model derived from a training dataset of RNA profiles for which the clinical diagnosis (seizure phenotype) is known. Once a model is identified with a high accuracy and sensitivity/specificity, it is then used to call a prediction on the test sample. The prediction may be a classifier value such as seizure/non-seizure, or a numerical value such as time following seizure. A multi factorial prediction model may be able to determine a factor that is greater than binary, for example FIA vs FTC vs GTC vs GIA seizure phenotype, or a recommended drug to treat the patient with. Prediction models are trained using common software packages and may involve linear/non-linear approaches or Machine learning Artificial intelligence algorithms.

In some embodiment, the diagnosis algorithm further provides a proposed treatment regimen based on the suspect's transcriptome profile, biographic information, person medical history and family medical history.

In some embodiments, determination of seizure occurrence is based on the expression level of a panel of seizure-associated biomarkers. As used herein, the term “seizure-associated biomarkers” refers to genes that are differentially expressed (e.g., either over-expressed or under-expressed) in seizure patients comparing to the expression level of the same markers in otherwise healthy individuals (e.g., in individuals who have not experienced and/or are not at risk of experiencing seizure). The seizure patient database maintains and updates a database of seizure-associated biomarkers, as well as a database of seizure-subtype associated biomarkers. As used herein, the term “seizure subtype-associated biomarkers' refers to genes that are either over-expressed or under-expressed in patients suffered from the corresponding subtype of seizure comparing to the expression level of the same markers in otherwise healthy individuals (e.g., in individuals who have not experienced and/or are not at risk of experiencing seizure). Seizure-subtype associated biomarkers include, but are not limited to, epileptic seizure-associated biomarkers and non-epileptic seizure-associated biomarkers.

In some embodiments, the overexpression or under-expression of seizure-associated biomarker/seizure-subtype associated biomarkers is determined with reference to the expression level of the same epileptic seizure-associated biomarker in an otherwise healthy individual. For example, a healthy or normal control individual has not experienced and/or is not at risk of experiencing epileptic seizure. As appropriate, the expression levels of the target ischemic seizure-associated biomarker in the healthy or normal control individual can be normalized (e.g., divided by) the expression levels of a plurality of stably expressed RNA reference expression blood profile biomarkers.

In some embodiments, the term “over-expression” refers to an expression level that is 50%, 100%, 200%, 500%, or 1000% greater than the reference expression level. In some embodiments, the term “under-expression” refers to an expression level that is less than 50%, 20%, 10%, 5%, or 1% of the reference expression level. In some embodiments, the reference expression level of a gene is the expression level of the gene in an individuals who has not experienced and/or are not at risk of experiencing seizure.

In some embodiments, the method of the present application further comprise the step of providing a recommendation for treatment and/or prevention regimes to a patient diagnosed as having a seizure or at risk of the occurrence of a seizure. Such recommendation may include medications and life-style adjustments (e.g., diet, exercise, stress) to minimize risk factors.

In some embodiments, the method of the present application is used as an adjuvant to traditional seizure diagnosis methods, such as EEG, MRI, and CT imaging. In some embodiments, the method of the present application further comprises the step of treating the seizure suspect with a seizure treatment.

Epileptic seizure can be treated by either medications, implanted devices, diet, surgery or a combination of these therapies. Most people are able to control the seizures caused by their epilepsy with medications called anti-epileptic drugs or AEDs. The type and severity of the seizure will determine what and how much medication is needed. The treatment for epileptic seizures may comprise: administering one or more drugs selected from the group consisting of brivaracetam, ezogabine, pregabalin, cannabidiol oral solution, felbamate, primidone, carbamazepine, fenfluramine, rufinamide, carbamazepine-XR, gabapentin, stiripentol, cenobamate, lacosamide, tiagabine hydrochloride, lamotrigine, clobazam, levetiracetam, topiramate, clonazepam, levetiracetam XR, topiramate XR, diazepam nasal, lorazepam, valproic acid, diazepam rectal, oxcarbazepine, vigabatrin, divalproex sodium-ER, phenobarbital, eslicarbazepine acetate, phenytoin and ethosuximide. One of ordinary skill in the art will understand the various ways known in the art to treat epilepsy and reduce epileptic seizures, and the methods disclosed herein are not limited to only those treatments listed herein.

Psychogenic non-epileptic seizures may be managed through a combination of psychological approach and pharmacological treatment.

Another aspect of the present application relates to a method of enhancing treatment of epileptic seizures. The method comprises the steps of obtaining a whole blood sample from a subject, wherein the subject is suspected to have experienced an epileptic seizure, preparing an RNA library from the whole blood sample and sequencing the RNA library, determining differential expression of a plurality of RNA sequences comprised within the RNA library, wherein the plurality of RNA sequences comprises non-coding RNA (ncRNA), creating a transcriptome profile based on the differential expression of the RNA sequences, comparing the transcriptome profile to a reference transcriptome profile for epileptic seizure, determining whether the subject suffered an epileptic seizure, based on the result of the comparing step, and treating the subject with a therapy for epileptic seizure if the subject is deemed to have suffered an epileptic seizure in the determining step.

In some embodiments, the reference transcriptome profile for epileptic seizure is a reference transcriptome profile having the same sex and race characterization as the subject.

In some embodiments, the therapy comprises the step of administering to the subject, an effective amount of one or more drugs selected from the group consisting of brivaracetam, ezogabine, pregabalin, cannabidiol oral solution, felbamate, primidone, carbamazepine, fenfluramine, rufinamide, carbamazepine-XR, gabapentin, stiripentol, cenobamate, lacosamide, tiagabine hydrochloride, lamotrigine, clobazam, levetiracetam, topiramate, clonazepam, levetiracetam XR, topiramate XR, diazepam nasal, lorazepam, valproic acid, diazepam rectal, oxcarbazepine, vigabatrin, divalproex sodium-ER, phenobarbital, eslicarbazepine acetate, phenytoin and ethosuximide.

Another aspect of the present application relates to a method for treating epileptic seizures in a subject. The method comprises the steps of generating a transcriptome profile from a blood sample obtained from the subject, confirming that the subject suffered an epileptic seizure by identifying epileptic seizure RNA signatures in the transcriptome profile of the subject, wherein the presence of epileptic seizure RNA signatures indicates that the subject suffered an epileptic seizure, and treating the subject confirmed of epileptic seizure with a therapy for epileptic seizure.

The present application is further illustrated by the following examples that should not be construed as limiting. The contents of all references, patents, and published patent applications cited throughout this application, as well as the Figures and Tables, are incorporated herein by reference.

### III. Data Analysis System and Program Product

As will be appreciated by one of skill in the art, method of the present application may be embodied as a data analysis system or program products. Accordingly, the method of the present application may take the form of data analysis systems or data analysis software, etc. Software written according to the present application is to be stored in some form of computer readable medium, such as memory, hard-drive, DVD ROM or CD ROM, or transmitted over a network, and executed by a processor.

One aspect of the present application provides a computer system for analyzing data from the transcriptome of a blood sample of a seizure suspect, and determining time of seizure occurrence, subtype of seizure, physiological status of the seizure suspect, potential treatment regimen and/or therapeutic efficacy. The computer system comprises a processor, and memory coupled to said processor which encodes one or more programs. The programs encoded in memory cause the processor to perform the steps of the above methods wherein the expression profiles and information about physiological, pharmacological and disease states of the seizure suspect are received by the computer system as input. The program encoded in memory also causes the computer to access the seizure patient database in order to perform analysis as described in the analysis step described above to generate an outcome.

Another aspect of the present application provides a server that harbors the database and the program for carrying out the method of the present application.

The present application is further illustrated by the following examples that should not be construed as limiting. The contents of all references, patents, and published patent applications cited throughout this application, as well as the Figures and Tables, are incorporated herein by reference.

### Examples

### Example 1. Application of Whole Transcriptome Analysis, Combined with Mathematical Modeling, Allows for Quantification of an Increased Numbers of RNAs in the Trancriptome

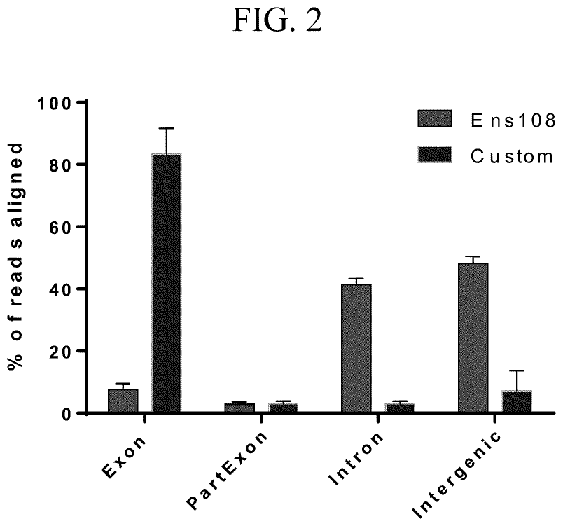

A pipeline has been developed to extract RNA from whole blood samples and perform whole transcriptome analysis, which assess whole transcriptome and not just protein encoding mRNAs. RNA was not primed with a polyT primer, reducing bias for polyA and 3′ transcripts. The RNA library generated from the extracted RNA were stranded. Custom annotation guides were created that allow quantification of novel RNAs that align to previously unannotated regions of the human genome. Such a custom blood transcriptome annotation increases the number of transcripts from 231957 to 269716. Quantification using this guide quantifies over 92% of the reads to such annotated regions vs. 30% using an Ensembl 108 based annotation guide (FIG. 2).

### Example 2. Blood Transcriptome Response to Electrically Evoked Seizure in Rats

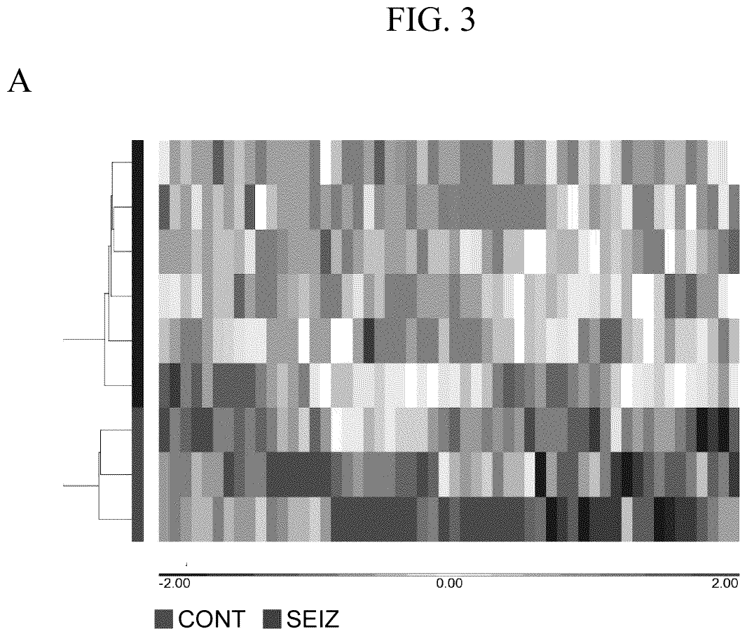

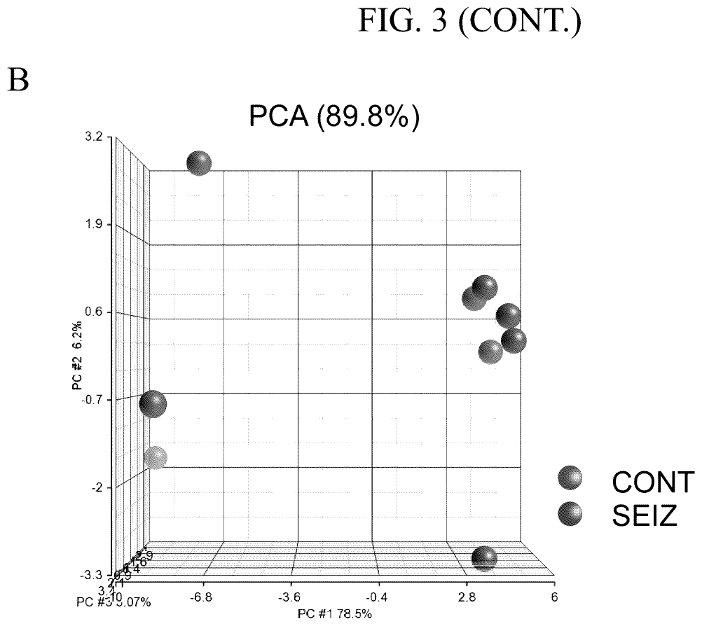

Rats without sedation were subjected to a short duration of electrical hippocampal stimulation that induces a seizure. The animals were sacrificed 24 h later, and blood collected for RNA sequencing. Differential gene expression revealed 122 genes with ±2.0-fold gene expression change passing a false discovery rate (FDR) p<0.05. Using differentially expressed RNAs for principal component analysis (PCA), the study shows the first three principal components account for over 94% of variance in the data. Blood RNA profiles obtained from animals that received an electrically evoked seizure are clearly different from the control non-seizure animals (FIG. 3).

Examples of the differentially expressed genes (identified by their Probeset ID) include, but are not limited to, Eif3g, Mapklip11, RT1-Cl, Eif4e2, Thoc3, Tmem243, Rela, Pidd1, Zfp772, Zfp282, Atp10a, Osbpl11, Srp72, Gent2, Slc24a3, Exoc2, Gnal, Ankrd54, RT1-T24-1, Orc4, Pdcd6, Fiz1, Stk19, Acp6, Xpr1, Ndufb4, Pphln1, Ppp2cb, Eif2d, Gsta4, Socs4, Fcsk, Tmem72, Nlgn2, Trmt2a, Nacc1, Hsd3b7, Mrps33, Cdc37, Xpnpep1, Stx2, Tp53i11, Lrrc8d, Ap3s2, Cnot8, Ufc1, Mrps26, Lrrc41, Ywhaz, Cnot2, Rbks, RT1-CE13, LOC288913, and Gemin7. Genomic location of the above-cited genes on rn6 reference genome can be determined via the Ensembl annotation guide v104. (http://ftp.ensembl.org/pub/release-104/gtf/rattus_norvegicus/Rattus_norvegicus.Rnor_6.0.104.gtf.gz).

### Example 3. Blood Transcriptome Response to EEG-Confirmed Seizure in Human

A study was performed to investigate blood transcriptome profiles in patients who have a seizure, whilst undergoing video EEG monitoring. Out of 175 admissions, the study approached 135 patients. Of these 77 were deemed ineligible, due to having had a seizure within 5 days (See exclusion criteria below). 17 declined, and 41 were deemed eligible and consented (30% of approached). Among these consented patients, 21 had a seizure (15% of patients approached), 2 withdrew and 18 left without a seizure occurring.

Video EEG monitoring: All patients undergo video EEG monitoring to record seizures or spells of interest. Patients were watched continuously by a bedside monitor or EEG technician for immediate reporting of any clinical event. EEG were interpreted by board certified epilepsy specialist. The epilepsy specialist confirmed the nature of the event, classified the event (epileptic seizure vs. PNES), recorded the event time of occurrence, and duration. For any events that could not be classified immediately, the blood collection procedure was initiated, and diagnosis was confirmed upon later review of EEG.

Sample Collection: Baseline blood samples were obtained (RNA, DNA and serum) upon admission at the beginning of video EEG monitoring. Once an EEG confirmed epileptic seizure or PNES is reported, blood samples were obtained at 4-6 hours, and 24 h post event termination (RNA and serum). The study arranged for the patient to visit the neurology clinic to obtain a 72 h and 7-day post-event blood sample. If the patient was unavailable, the study arranged to visit the patient and obtain a blood sample. In cases of multiple seizures, the study obtained the 6 h blood sample post 1st seizure, and 6 h, 24 h, 72 h and 7 days after last seizure. All samples received a unique identifier and were couriered to the laboratory facility (MSM). Blood samples and isolated RNA were stored in a secured −80° C. freezer. PAXgene tube samples were not split.

Controls: Each patient acted as their own control (baseline blood sample). Additionally, patients with EEG confirmed PNES acted as a second control. Finally, some patients who are recruited but do not have an EEG verified seizure, were a third control. No seizure controls (non-PNES) had two samples drawn (baseline and end of study/discharge).

RNA-Seq Library Preparation: Blood (3.0 mL) was drawn into PAXgene blood collection tubes, and processed using a QiaCUBE (Qiagen). RNA quality was verified using a Bioanalyzer (RNA chip, Agilent Bioanalyzer 2100). Only samples with an A260/A280 ratio >2. 0 and a 28S/18S RNA ratio >5 was subjected to further analysis. RNA yield from whole blood was 3-8 μg/3 mL of whole blood (not shown). ERCC “spike in” control RNA assessed library preparation QC. RNA-Seq Libraries were prepared for the Ion Torrent sequencer using the Ion Total RNA-Seq Kitv2. The libraries were verified with a Tapestation (DNA Nano Kit, Agilent), and loaded on chips using an IonChef. Samples were run on Ion 540 chips in batches of 4-8, across two-three chips to give an estimated read depth of 40 million reads/sample. Seq data were analyzed to ensure appropriate base and GC content distribution. The study obtained approx. 40M aligned reads/blood sample, based on depth analysis. Some samples were also be subjected to long read analysis using the Oxford Nanopore platform to verify splicing changes following seizure using a cDNA kit. Samples were multiplexed and run on Promethion chips to achieve 50M reads/sample.

Data Alignment: Sequencing data was aligned to the human reference genome (Grch38) using STAR and Bowtie2 (on local Dell cluster) or minimap2/hisat2 for long read data. RNA-Seq data files (BAM files) were used to generate gene, transcript and exon expression values. Once gene read values were determined, they were transferred to a local encrypted database for storage (QNAP TVS1828T). Sequencing data were stored locally prior to upload to NIH. The study maintained a de-identified database of clinical phenotype data alongside each transcriptome.

The study sequenced 26 sets of seizure samples, consisting of baseline, 6 h post event and 24 h post event. The study lost 4 patients worth of data due to RNA extraction issues (failed QC). RNA was subjected to RNA-Sequencing using the Ion Torrent Platform (ThermoFisher). RNA sequencing data (reads) were aligned to the human genome (Grch38) and analyzed using Partek Genomics Studio. Gene expression and transcript usage was determined using linear models following correction for batch (in linear model).

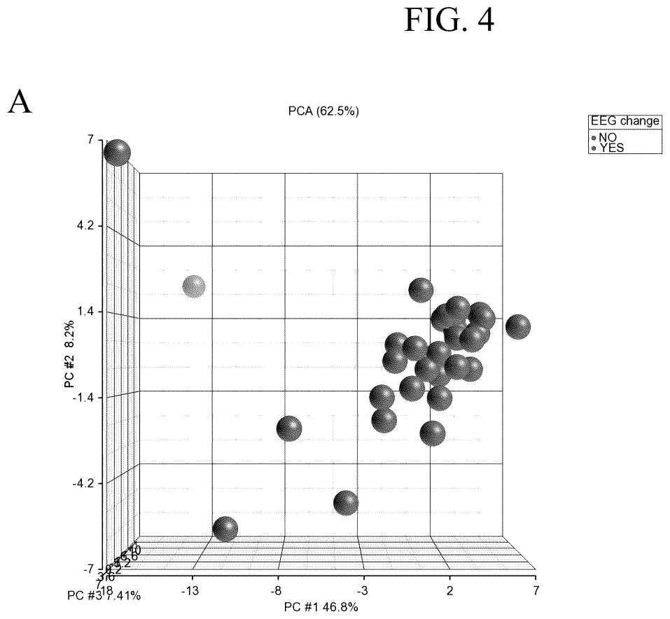

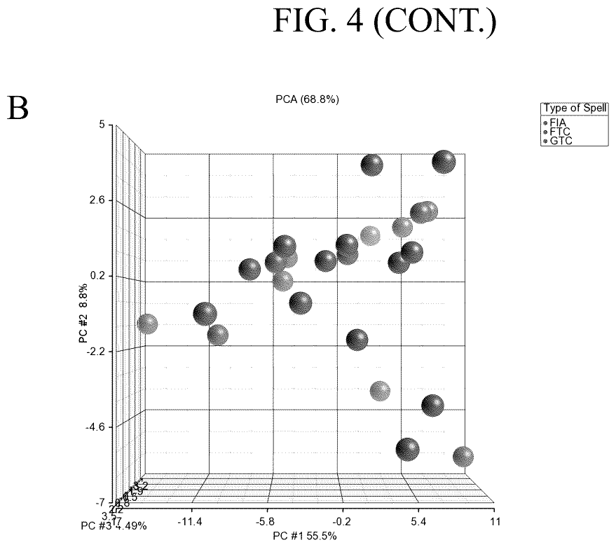

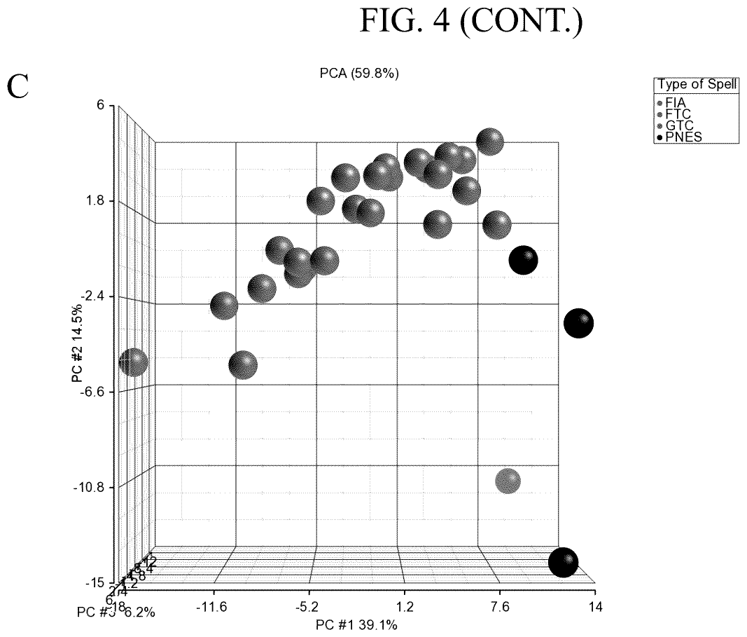

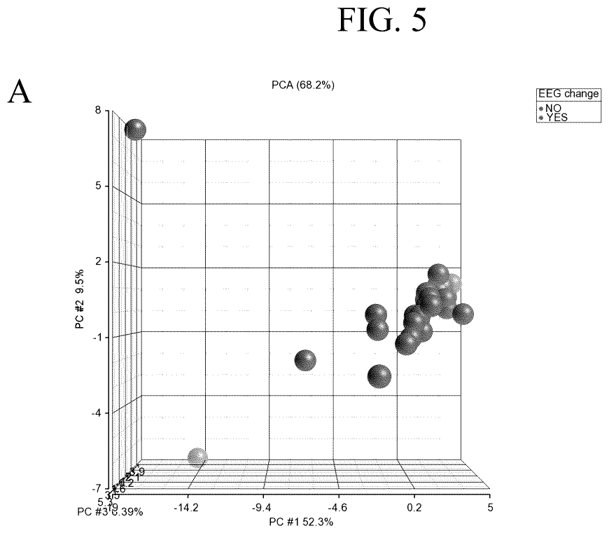

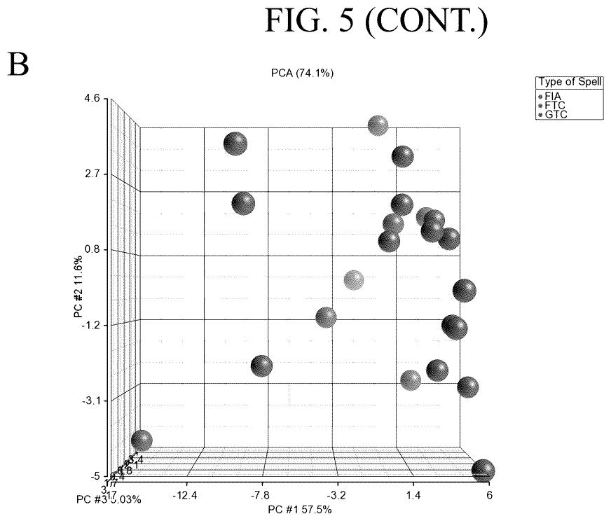

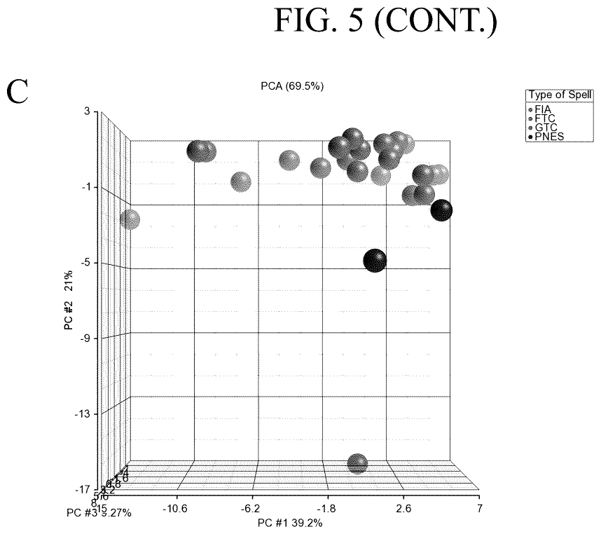

Principle component analysis of differentially expressed genes shows clear separation of EEG confirmed seizure from non-seizure (FIG. 4, Panel A) and PNES samples from other seizures (FIG. 4, Panel B) at 4-6 h post seizure. PCA analysis also shows separation of PNES samples from other seizure types (FIG. 5, Panels A and C), as well as separation of different type of seizures (FIG. 5, Panel B) at 24 h post seizure (FIG. 5, Panel A).

Interestingly using gene expression analysis, focal to bilateral tonic clonic (FTC) seizures show furthest separation (PC1) from other seizure types. This indicates gene expression changes persist up to 24 h following a seizure.

Examples of the differentially expressed genes (identified by their Probeset ID) in seizure vs. non-seizure patients at 4-6 h post seizure (FIG. 4, Panel A) include, but are not limited to, TMEM179B, ID2, DHX8, ENSG00000218426, PTCD2, RPL10P16, THOC5, TYW5, ENSG00000232626, RPLPOP6, ITM2A, F2R, C11orf58, PACS1, JUND, ENSG00000283907, ERH, MBD3, SLC25A30, PRDX3, YWHAZP3, RPL4P4, YBX1P10, HDAC9, GPX4, MESD, AHCTF1P1, RPL13AP5, RABIF, ARL6IP5, ENSG00000218175, KDM2B, SNX17, GPX1, EIF2S3B, ENSG00000270066, LANCL1, PTGER4, ANXA7, MED29, PTGER2, EEF1A1P11, SP1, MFHAS1, ENSG00000244313, GSTP1, EEF1A1P4, TRBC1, PARP6, PCID2, PIGV, FAN1, INPP5F, UTP23, FECH, FBX09, RPS2P46, ANKRD10, HPCAL1, SLC35E1, LY75-CD302, DUSP7, PSMG2, TRIM24, MRS2, KLF2. Genomic location of genes on hg38 reference genome can be determined via the Ensembl annotation guide v109. (https://ftp.ensembl.org/pub/release-109/gtf/homo_sapiens/Homo_sapiens.GRCh38.109.gtf.gz).

Examples of the differentially expressed genes (identified by their Probeset ID) in different type of seizures at 4-6 h post seizure (FIG. 4, Panel B) include, but are not limited to, BORCS8, CAD, DERL1, ENSG00000291067, MT-CO3, TMEM219, ABCA7, AKT3, ALPL, APMAP, ARL11, ARRB2, ASB1, CTNNA1, CYB5R4, DHX34, DNAJB6, DOCK5, EEF1G, ELAC2, ENSG00000290791, FAM8A1, FCHO2, FPR2, GCA, GLTID1, GMPR2, HEBP2, HYCC2, IL10RB, JAML, KCNJ15, KREMEN1, LAT2, LBH, LINC01127, LYAR, MPZL3, MSL1, NADK, NELFCD, NPEPPSP1, PELI1, PLXNC1, PPP2R5A, PSMC4, RNF149, RPL13A, RPL37A, SDHAF2, SIGLEC9, SLC11A1, SLC16A3, SLC9A8, SNORD110, SNX10, SOS2, TMEM120A, TMEM71, TMLHE, TNFRSF10C, TRBC2, TREML2, TSEN34, VNN3P, WRN, ZBTB38, ZNF516, CWF19L1, EIFIAD, GNB4, HNRNPA1P10, NSRP1, RPL13AP5, RPL6P27, RPL7P1, SP140L, TIGIT, DHPS, MBTPS1, PPIE. Genomic location of genes on hg38 reference genome can be determined via the Ensembl annotation guide v109. (https://ftp.ensembl.org/pub/release-109/gtf/homo_sapiens/Homo_sapiens.GRCh38.109.gtf.gz).

Examples of the differentially expressed genes (identified by their Probeset ID) in seizure vs. non-seizure patients at 24 h post seizure (FIG. 5, Panel A) include, but are not limited to, RN7SKP176, RN7SKP255, PTGER4, RN7SL396P, TAOK1, DEAF1, YBX1P10, DAP, RN7SKP203, TVP23C, ZNF687, UBBP4, ZBTB7A, PGLS, ORAII, HBZP1, DCTN2, AGPAT1, FAM172A, PRELID3B, LLGL2, RNVU1-31, D2HGDH, IQCN, ACTBP2, RN7SL674P, PTPA, ZNF189, DPEP2, SNHG32, RN7SKP71, EDEM3, TSC22D2, SMURF1, ENSG00000283907, MAPIS, USP34, MTHFS, HBA1, RN7SL3, SF3B5, EPC1, MAN2C1, PCBP2P2, HBA2, MRTFB, SEC14LIP1, HK1, CALM3, RPS2P46, RABGEF1, AHCYL1, PSMC1, TECPR1, YBX1P1. Genomic location of genes on hg38 reference genome can be determined via the Ensembl annotation guide v108. (https://ftp.ensembl.org/pub/release-109/gtf/homo_sapiens/Homo_sapiens.GRCh38.109.gtf.gz).

Examples of the differentially expressed genes (identified by their Probeset ID) in different type of seizures at 24 h post seizure (FIG. 5, Panel B) include, but are not limited to, RBM7, ATP11B, DNAJC3, FAM91A1, FAR1, FBXL5, FOXN2, GCA, GLIPR2, HSDL2, HYCC2, ITM2B, LMBRD1, LPGAT1, MANSC1, MTMR6, NABP1, PELI1, PIP4P2, PSTPIP1, PTEN, RAB10, RAB5IF, RGL2, RNF149, RRM2B, SNHG1, SNHG32, SNORA31, SNORA81, SNORD11, SNORD110, SNORD25, SNORD27, SNORD30, SNORD35B, SNORD42B, SNORD50B, SNORD52, SNORD56, SNORD58B, SNORD6, SNORD60, SNX10, SPOPL, SULT1B1, TLR1, TMLHE, UBE2A, UBE2D1, WIPI1, ACTR8, CDC123, GANAB, PCYOX1, RPL36A, ZNF827, CNEPIR1, MYCBP. Genomic location of genes on hg38 reference genome can be determined via the Ensembl annotation guide v108. (https://ftp.ensembl.org/pub/release-109/gtf/homo_sapiens/Homo_sapiens.GRCh38.109.gtf.gz).

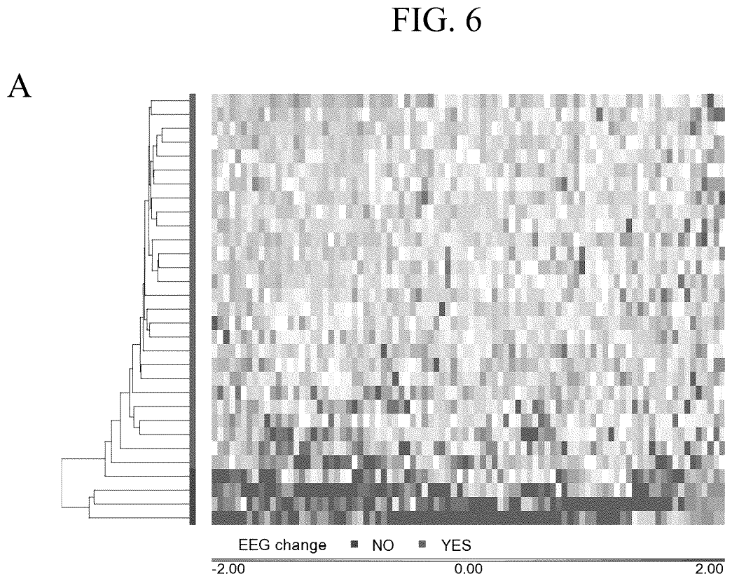

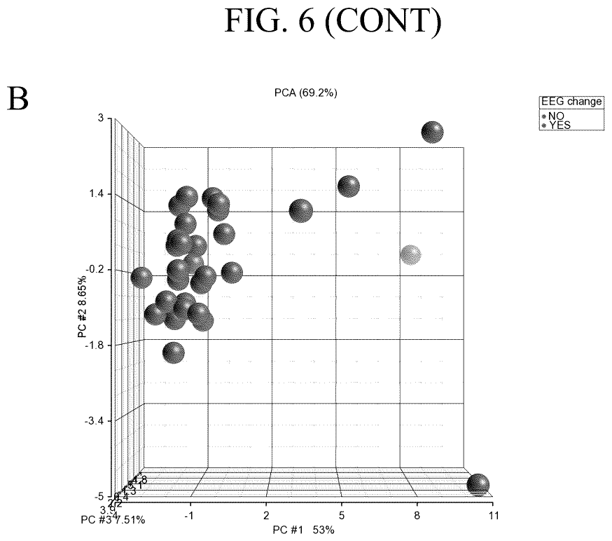

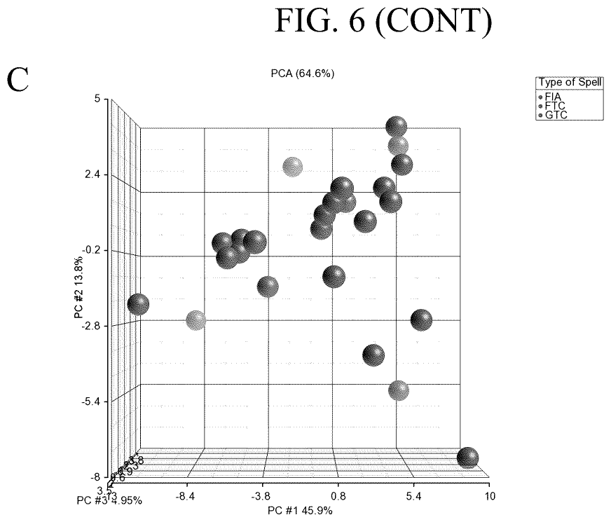

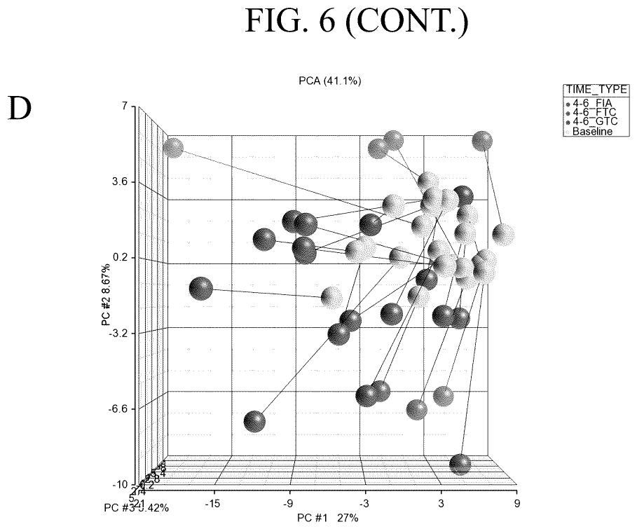

Analysis of transcript usage show differential splicing of RNA occurs in blood following seizure. The differential splicing patterns (as deemed by different isoform usage) can also identify a patient who has suffered a seizure, as determined using hierarchical clustering analysis (FIG. 6, Panel A) and PCA (FIG. 6, Panels B-D). This study found that 88 genes are alternatively splice at 4-6 h post seizure, to define a PNES seizure vs EEG confirmed seizure, and 64 genes show alternative splicing at 6 h to distinguish seizure types. Principal component analysis shows a clear separation of data along two main components. Transcript analysis reveals a striking separation of patients with focal tonic clonic (FTC) seizures (FIG. 6, Panel B). This suggests that splicing may enable a differentiation of seizure type, suggesting rapid direct RNA-seq approaches may be useful for identifying patients who have suffered a GTC seizure in a point of care assay.

Examples of the differentially expressed transcripts (identified by the Probeset ID of the corresponding genes) in seizure vs. non-seizure patients at 4-6 h post seizure (FIG. 6, Panels A and B) include, but are not limited to, ENST00000397797>HBA1, ENST00000510187>SQSTM1, ENST00000695862>CDC42, ENST00000373298>ITM2A, ENST00000652583>ZFP36, ENST00000530945>CFL1, ENST00000417053>ENSG00000232626, ENST00000507943>BODIL1, ENST00000693666>GOLGA3, ENST00000503742>UBE2D3, ENST00000553630>CALM1, ENST00000544690>TYROBP, ENST00000250896>MKNK2, ENST00000427401>ZNF737, ENST00000273223>RPL32, ENST00000228136>C11orf58, ENST00000415292>YWHAZP3, ENST00000394878>RPLPOP6, ENST00000492974>ENSG00000218426, ENST00000695948>TRERF1, ENST00000319211>F2R, ENST00000325000>RPL10P16, ENST00000444752>YBX1P10, ENST00000393238>TMCC1, ENST00000273258>ARL6IP5, ENST00000422486>RPL4P4, ENST00000602755>ENSG00000270066, ENST00000367262>RABIF, ENST00000638356>ENSG00000283907, ENST00000202677>RALGAPA2, ENST00000420969>AHCTF1P1, ENST00000394432>RASGRP2, ENST00000333219>ING1, ENST00000439189>RPL13AP5, ENST00000398606>GSTP1, ENST00000304414>ARL6IP1, ENST00000299927>ZNF592, ENST00000372919>ANXA7, ENST00000276282>MFHAS1, ENST00000405359>ENSG00000218175, ENST00000380680>UBL3, ENST00000421246>CDC42P6, ENST00000467930>ENSG00000244313, ENST00000252818>JUND, ENST00000376802>HLA-A, ENST00000245457>PTGER2, ENST00000162749>TNFRSF1A, ENST00000415278>EEF1A1P11, ENST00000298510>PRDX3, ENST00000457354>TMX1, ENST00000508832>MALAT1, ENST00000359165>EEF1A1P4, ENST00000607003>SAP18, ENST00000633705>TRBC1, ENST00000349213>ARID4B, ENST00000490163>UBXN4, ENST00000554465>SRSF5, ENST00000234310>PPP3R1, ENST00000484897>RPSAP58, ENST00000458332>RPS2P46, ENST00000511164>RAC1P2, ENST00000377474>KCTD12, ENST00000382873>FECH, ENST00000676189>ACTB, ENST00000683488>ATM, ENST00000307808>AFF1, ENST00000498502>MBNL1, ENST00000615892>PPP1R18, ENST00000440859>CBLL1, ENST00000673846>BIRC3, ENST00000392563>GRB2, ENST00000670229>FMNL1-DT, ENST00000462885>RPL18AP3, ENST00000650601>HBD, ENST00000363046>RMRP, ENST00000377795>CD74, ENST00000498661>ORAI2, ENST00000392754>FAM53B, ENST00000248071>KLF2, ENST00000635620>DENND4A, ENST00000335007>PPP1CC, ENST00000274031>SETD7. Genomic location of transcripts on hg38 reference genome can be determined via the Ensembl annotation guide v109. (https://ftp.ensembl.org/pub/release-109/gtf/homo_sapiens/Homo_sapiens.GRCh38.109.gtf.gz). Examples of the differentially expressed genes (identified by the Probeset ID of the corresponding genes) in different seizure type at 4-6 h post seizure (FIG. 6, Panel C) include, but are not limited to, ENST00000330720>KDELR1, ENST00000361624>MT-CO1, ENST00000362079>MT-CO3, ENST00000519638>ERLIN2, ENST00000581241>CSNKID, ENST00000694951>IL17RA, ENST00000216146>RPL3, ENST00000217456>APMAP, ENST00000233143>TMSB10, ENST00000250360>SIGLEC9, ENST00000265085>CPEB4, ENST00000272519>RALB, ENST00000290200>IL10RB, ENST00000295317>RNF149, ENST00000326648>ZNF609, ENST00000329251>EEF1G, ENST00000339569>MSL1, ENST00000356864>TNFRSF10C, ENST00000367075>EZR, ENST00000368885>AMD1, ENST00000369535>NRAS, ENST00000374391>ALOX5, ENST00000374840>ALPL, ENST00000389120>RNF20, ENST00000391857>RPL13A, ENST00000395323>LBH, ENST00000395762>IL4R, ENST00000396946>CARD11, ENST00000397708>MCM3AP, ENST00000418596>HYCC2, ENST00000443185>ZNF516, ENST00000466254>TRBC2, ENST00000483722>TREML2, ENST00000487445>GCA, ENST00000492413>SLC11A1, ENST00000513001>ACSL1, ENST00000520106>SLA, ENST00000547026>ZNF641, ENST00000550811>TUBA1A, ENST00000561488>XPO6, ENST00000607197>HEBP2, ENST00000608318>ENSG00000290791, ENST00000613060>SLC6A6, ENST00000624694>HRH2, ENST00000627215>LINC01127, ENST00000672722>ARHGAP35, ENST00000673794>VNN3P, ENST00000678703>EEF1A1, ENST00000703699>GNB1, ENST00000706367>ACSL1, ENST00000398253>ZFP90, ENST00000439151>NSD1, ENST00000444945>HNRNPA1P10, ENST00000468161>RPL7P1, ENST00000526668>RPL8, ENST00000552951>NOPCHAP1, ENST00000683488>ATM, ENST00000683697>MYCBP2, ENST00000696270>TNRC6C, ENST00000646122>DDX3X, ENST00000360902>TRRAP, ENST00000367536>NCF2, ENST00000426496>PRRC2C, ENST00000456586>RPL37A. Genomic location of transcripts on hg38 reference genome can be determined via the Ensembl annotation guide v109. (https://ftp.ensembl.org/pub/release-109/gtf/homo_sapiens/Homo_sapiens.GRCh38.109.gtf.gz)

Examples of the differentially expressed transcripts (identified by the Probeset ID of the corresponding genes) in seizure vs. non-seizure patients at 24 h post seizure include, but are not limited to, ENST00000478004>PSMF1, ENST00000363673>RN7SKP176, ENST00000487791>HBA1, ENST00000484216>HBA2, ENST00000363442>RN7SKP255, ENST00000471086>RN7SL396P, ENST00000248437>TUBA4A, ENST00000472694>HBA1, ENST00000482565>HBA2, ENST00000230895>DAP, ENST00000444752>YBX1P10, ENST00000363618>RN7SKP203, ENST00000379419>PDE7A, ENST00000356444>SEMA4D, ENST00000354915>HBZP1, ENST00000598034>GMFG, ENST00000487283>ZAP70, ENST00000322357>ZBTB7A, ENST00000462494>ACTB, ENST00000605806>RNVU1-31, ENST00000409064>KDM3A. Genomic location of transcripts on hg38 reference genome can be determined via the Ensembl annotation guide v109. (https://ftp.ensembl.org/pub/release-109/gtf/homo_sapiens/Homo_sapiens.GRCh38.109.gtf.gz).

Examples of the differentially expressed genes (identified by the Probeset ID of the corresponding genes) in different seizure type of seizures at 24 h post seizure include, but are not limited to, ENST00000336332>ZXDC, ENST00000338272>TMEM167B, ENST00000377461>CARD8, ENST00000492741>PSPC1, ENST00000233612>GCA, ENST00000251810>RRM2B, ENST00000260130>SDCBP, ENST00000280098>SPOPL, ENST00000295317>RNF149, ENST00000332180>WASHC4, ENST00000334398>TMLHE, ENST00000335181>PKM, ENST00000355356>MIER1, ENST00000358912>PELI1, ENST00000362607>SNORA31, ENST00000363981>SNORD27, ENST00000364884>SNORD52, ENST00000364995>SNORD50B, ENST00000365607>SNORD25, ENST00000366997>LPGAT1, ENST00000369856>FLNA, ENST00000374832>ALPL, ENST00000384693>SNORD30, ENST00000395788>SNHG32, ENST00000413522>SNORD56, ENST00000420627>FLNA, ENST00000432261>RNF24, ENST00000437150>GCA, ENST00000443799>GAS5, ENST00000458893>SNORD42B, ENST00000466047>MCMBP, ENST00000487445>GCA, ENST00000527983>HSPA8, ENST00000538654>SNHG1, ENST00000550811>TUBA1A, ENST00000620947>MCL1, ENST00000649266>ITM2B, ENST00000650315>OGFRL1, ENST00000688158>PTEN, ENST00000702934>GAS5, ENST00000339569>MSL1, ENST00000313367>OSBPL3, ENST00000357164>GM2A, ENST00000368003>UFC1, ENST00000376406>MDC1, ENST00000613780>MIAT, ENST00000673822>DDHD1, ENST00000677965>EIF3E, ENST00000700035>ARHGAP35, ENST00000703700>GNB1, ENST00000480503>MYO1G. Genomic location of transcripts on hg38 reference genome can be determined via the Ensembl annotation guide v108. (https://ftp.ensembl.org/pub/release-109/gtf/homo_sapiens/Homo_sapiens.GRCh38.109.gtf.gz).

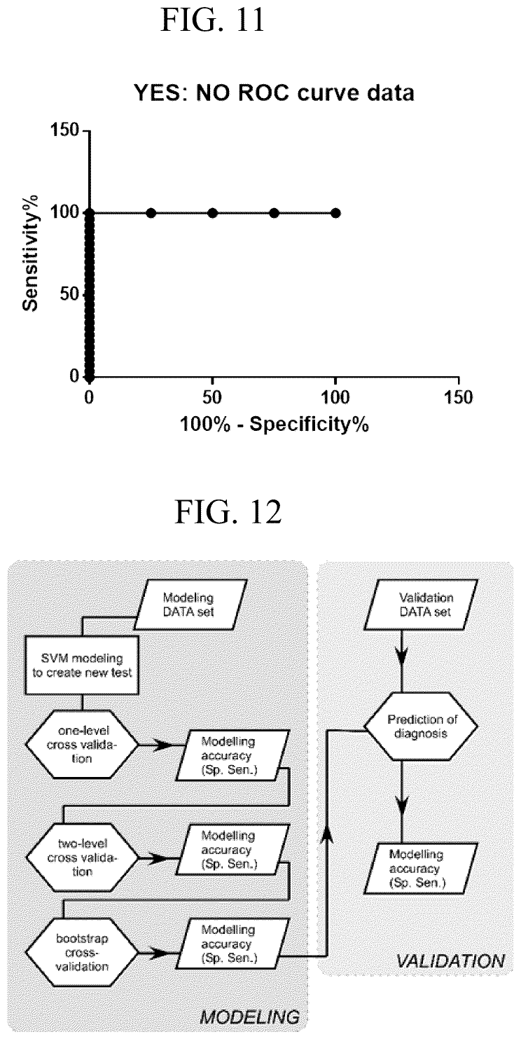

Finally, the study used K-Nearest Neighbors (KNN) and support vector machine (SVM) modeling to identify potential models with the ability to predict the occurrence of a seizure. Using the 6 h data, SVM 6 h prediction models performed best, with an accuracy of 78.5% accuracy (80% sensitivity (SN) and 70% specificity (SP)) vs KNN (1 nearest neighbor) accuracy of 77% SN 75% SP 69%. Using ROC analysis of PC1 (FIG. 4, Panel A), the study can identify seizure occurrence with accuracy of 100% (FIG. 11). These data show that patterns of gene expression and transcripts in blood have utility as a molecular biomarker to identify if a patient has suffered a seizure.

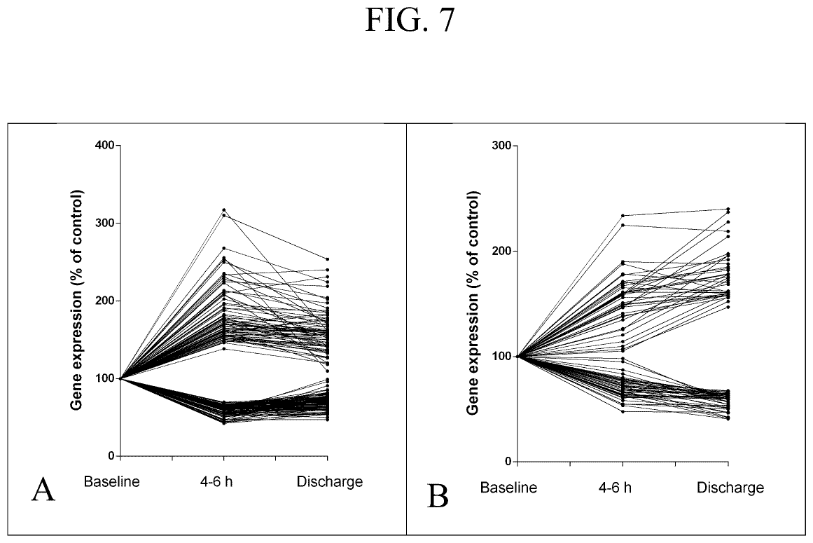

FIG. 7 shows that gene expression patterns change over time in patients with EEG-confirmed seizure and in patients with no EEG change seizure (PNES). Panel A: Time course of genes identified at 4-6 h time point as being differentially expression in patients with EEG change seizure. Panel B: Time course of genes identified at 24 h time point as being differentially expression in patients with EEG change seizure. Panel C: Time course of genes identified at 4-6 h time point as being differentially expression in patients with no EEG change seizure (PNES). Panel D: Time course of genes identified at 24 h time point as being differentially expression in patients with no EEG change seizure (PNES). Analysis reveals 187 genes show differential expression vs baseline control following a seizure. In contrast, 61 genes show differential expression in PNES patients at 4-6 h and 24 h (e.g., at discharge) post event compared to baseline. These data show some RNAs are temporally expressed following a seizure, and that temporal patterns of gene expression may enable the determination of when a seizure occurred in the past.

Examples of the differentially expressed genes (identified by their Probeset ID) in EEG confirmed seizure groups vs baseline at 4-6 h post seizure (FIG. 7, Panels A and B) include, but are not limited to, ADAMTSL4, ADH5, AQP9, BCL2A1, BCL6, BDH1, BIRC3, BTNL8, Clorf52, CAMK4, CCNJL, CD200R1, CEACAMI, CEBPB, CEP290, CHSY1, CLEC4D, CLEC4E, CR1, CRISPLD2, DCAF13, DHRS13, DPP4, EEF1A1P22, ENSG00000218426, ENSG00000238035, ENSG00000241860, ENSG00000259600, ENSG00000274272, ENSG00000288932, ENSG00000289013, ENSG00000290021, ENSG00000290385, ENSG00000290585, ENSG00000290937, ENSG00000291215, ENSG00000291221, F5, FAM157C, FAM157D, FCAR, FKBP5, FKTN, FOSL2, FUT7, GDPD5, GRAP, GSEC, GTF2IRD2B, HSPA6, ILIR1, IRAG1, KCNE1, KCNJ15, KPNA5, LEO1, LIMK2, LINC01001, LINC01002, LIPN, LMNB1, LYRM7, MANIC1, MMP25, MMP9, MTCYBP23, MTND5P32, MXD3, NAIP, PCDH9, PELATON, PFKFB3, PFKFB4, PGS1, PHC2, PLEKHA8, PLIN5, PPA1, QPCT, RALGAPA1P1, RNF157, RPL13AP25, RPL13AP5, RPL3P4, RPL4P4, RPS23P8, RPS26, RRP1B, S100A8, S100P, SCARF1, SFXN1, SLAMF1, SLC12A9, SLC37A3, SLC39A10, SNORD14D, TBKBP1, THEMIS, TMEM263, TMX2, TOMM7, TP53111, TRBC1, TXNDC15, TYW1, UBIAD1, VPS9D1, WDFY3, ZBTB5, ZC3H12D, ZNF260, ZNF480, ZNF568, ZNF7, ZSCAN12, ACP6, ADM, AK3, ALG2, ALG5, ANK3, ATP1B3, B4GALT5, BASP1, CCDCl41, CD8B, CLEC4A, CUTA, DBP, DMAP1, ECI2, ENSG00000249624, ENSG00000291066, GK, HAUS4, IFT80, KLHDC4, KREMEN1, LILRA5, LINC01550, LSG1, MRTFB, NAMPTP1, NDUFA12, NLRC4, OSCAR, PGLYRP1, PLSCR1, POLA1, RYK, SELENOW, SIGLEC5, SMG1P1, ST20, TESPA1, TMEM18, TNFRSF25, WDR27, ZNF441, ZNF559, ZNF594, ACSL1, ADGRG3, AKRIB1, ALPL, ANXA3, BCL3, CNTNAP3C, CYSTM1, DYSF, ENSG00000290890, GNG10, GOLT1B, HRH2, LINC01127, LRG1, MGAM, MGAM2, MTARC1, PROK2, S100A12, SIPA1L2, SLC11A1, TGFA, TLR5, TRPM2. Genomic location of genes on hg38 reference genome can be determined via the Ensembl annotation guide v109. (https://ftp.ensembl.org/pub/release-109/gtf/homo_sapiens/Homo_sapiens.GRCh38.109.gtf.gz).

Examples of the differentially expressed genes (identified by their Probeset ID) in EEG confirmed seizure groups vs baseline at 24 h post seizure (FIG. 7, Panels C and D) include, but are not limited to, ENSG00000286813, FBX09, AHCYL1, ALOX12-AS1, ARHGAP19, ARID3B, ATP5MC2, C4orf3, C6orf47, CCDC28A, CHPF2, CTPS1, D2HGDH, DCTN2, DPEP2, DPH3, DST, EIF4ENIF1, ENSG00000289474, ESS2, FAM20B, FAM98A, GAS5, GDE1, GTPBP6, HERC6, IQCN, LIMK1, MTERF4, NELFE, NSUN3, PGLS, PPP2CA, PSMC1, RABGGTB, RANBP9, RBM18, RN7SKP255, RNVU1-31, SLC19A1, SMC3, SNHG29, SNHG32, SNORD100, SNORD30, SNORD34, SNORD41, SNORD5, SNORD69, SNORD91B, STRN, STX17, TDP2, TECPR1, TOP3A, TTF1, TVP23C, USP16, USP33, WDR6, ZNF766. Genomic location of genes on hg38 reference genome can be determined via the Ensembl annotation guide v109. (https://ftp.ensembl.org/pub/release-109/gtf/homo_sapiens/Homo_sapiens.GRCh38.109.gtf.gz).

### Example 4. Principal Component Analysis (PCA) Showing Discriminant Whole Blood RNA Profiles of Seizure and Non-Seizure Stroke Mimics

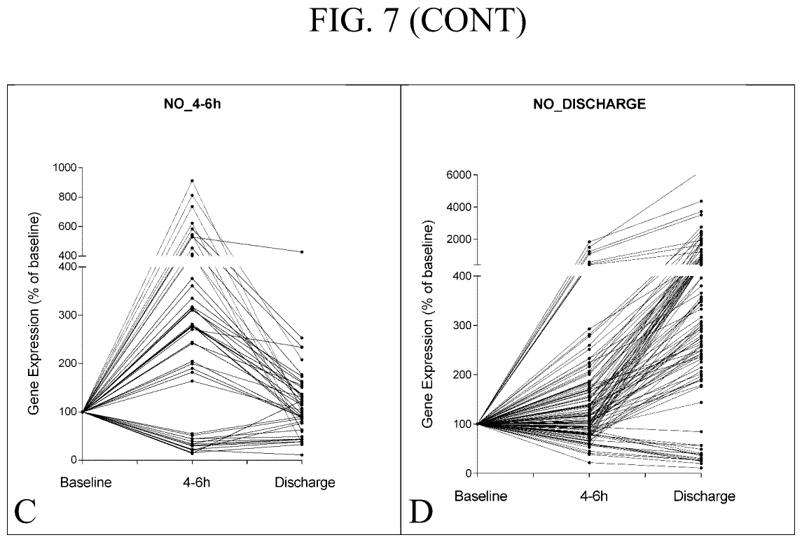

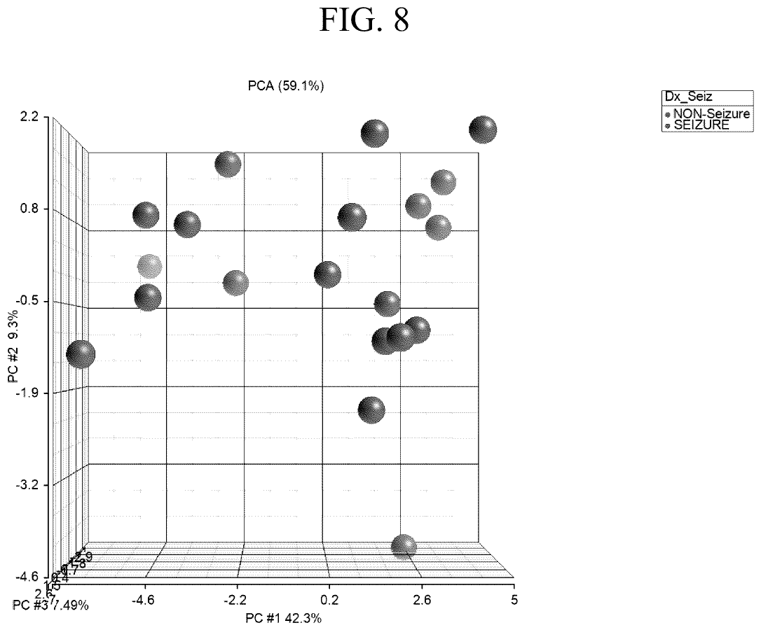

Transient ischemic attacks (TIA) may sometimes be misdiagnosed as a seizure. An analysis of 200 blood samples collected from stroke patients shows that patients who are designated stroke mimics, but later adjudicated to have suffered seizure, have distinct blood RNA profiles from other cause stroke mimics (FIG. 8). This finding suggests that patients diagnosed with seizure may have RNA profiles that are different from those diagnosed TIA.

### Example 5. Effect of Sex and Race on Seizure Profiles

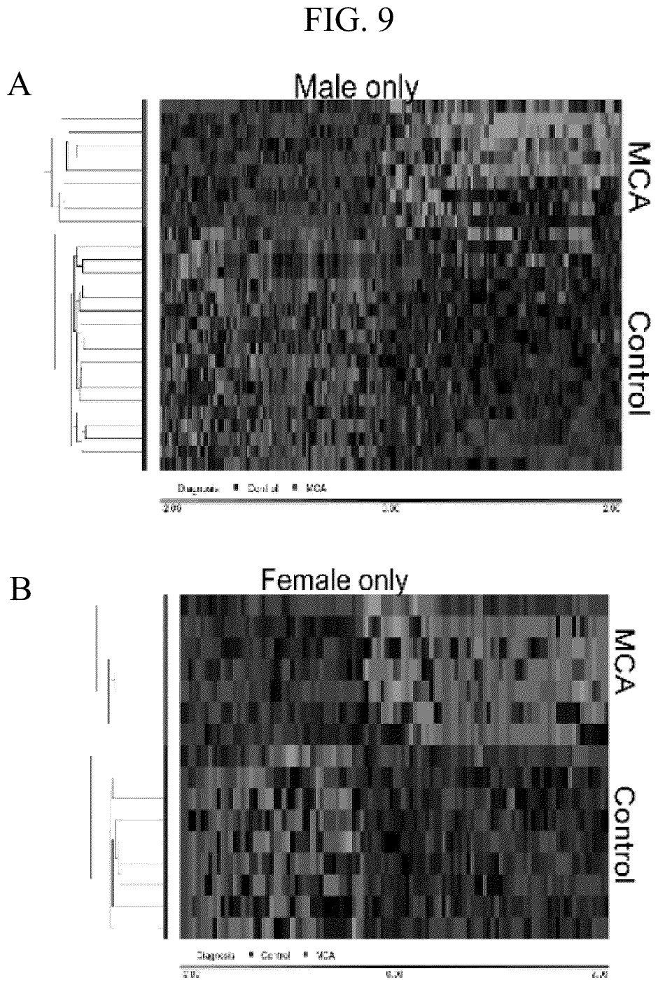

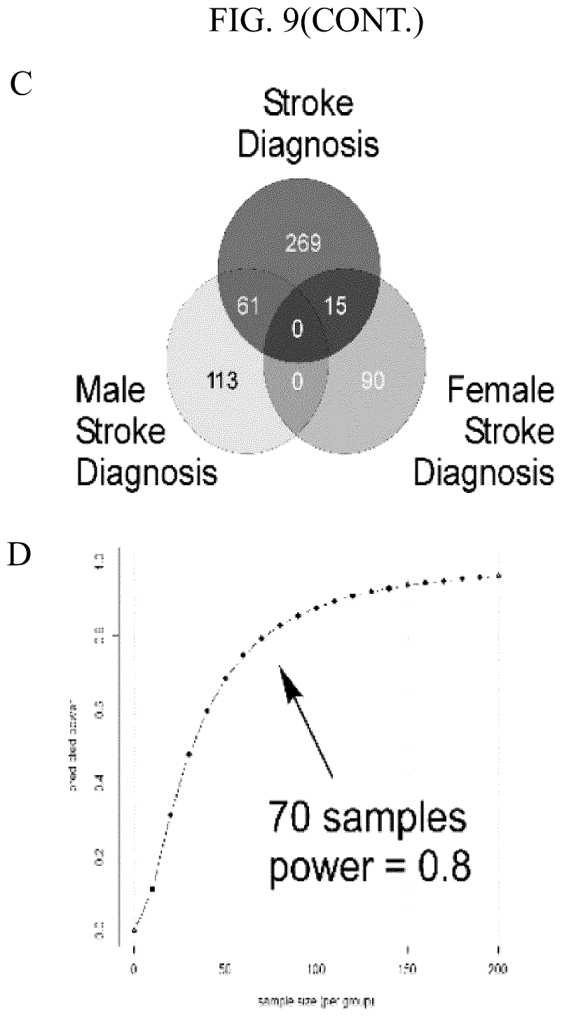

The above-described study also investigated the impact of correcting for sex and race on the gene expression responses to various seizure subtypes. As can be seen in FIG. 9, Panels A and B), in a stroke study, investigating sex-specific RNA changes following stroke revealed different genes are regulated following a stroke in males and female patients. Venn diagram shows overlap of exons in models identifying mixed gender, or sex specific changes following MCA (FIG. 9, Panel C). Power analysis modeling of sex specific data (female) showed 2-fold changes with FDR 0.05 (FIG. 9, Panel D, modeled in SPAA).

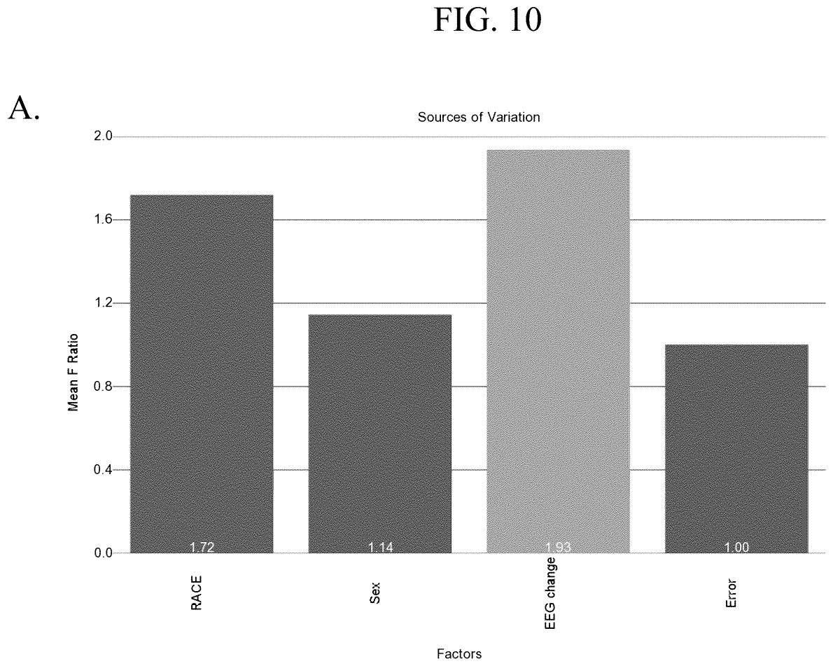

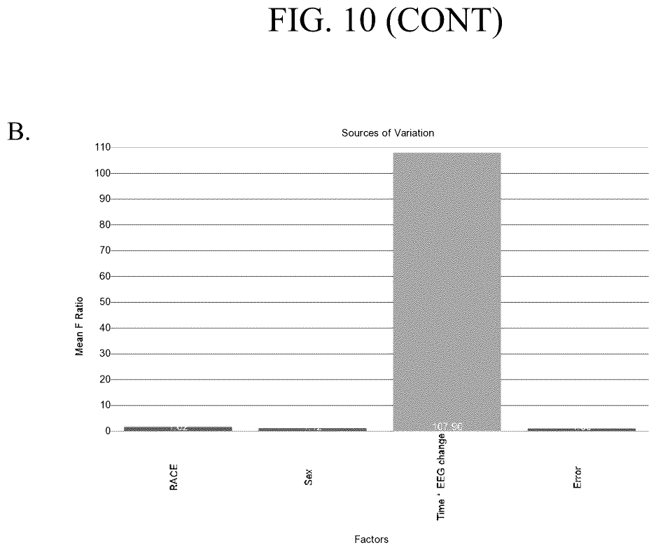

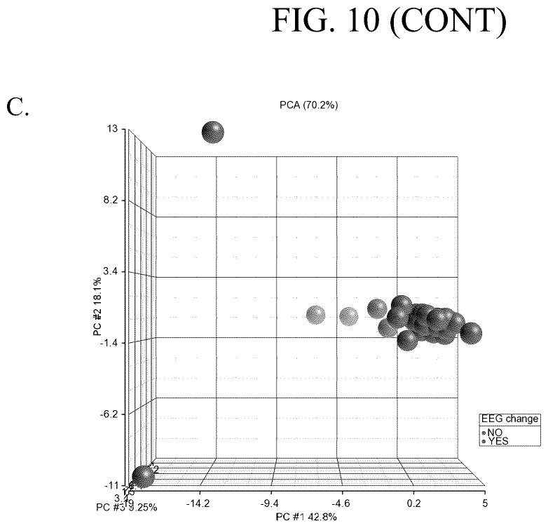

FIG. 10 shows correcting for sex and race reduces the number of differentially expressed genes identified in blood following seizures. Data from 4-6 h samples were also subjected to differential expression analysis, with and without correction for race and gender. Genes showing significant 1.5-fold changes were subjected to Principal component analysis. Panels A and B show sources of variation following 5 linear modeling of the data using 5 contrast variables. Panel C shows corrected differentially expressed genes in seizure vs non-seizure 4-6 h group.

Examples of the differentially expressed genes (identified by their Probeset ID) in seizure vs. non-seizure patients at 4-6 h with race and sex correction (FIG. 10, Panel C) include, but are not limited to, TTLL3, GPX1, GTF3C1, CYTOR, RNASEL, SEPHS2, FECH, PACS1, RAB5IF, DUSP7, MYNN, FBXO9, TRIM24, DHX8, F2R, SNX17, ERF, HNRNPKP4, GTF2IRD2, POGLUT1, DUT, TMEM30A, CBLL1, ENSG00000291117, ADAM9, TSNAX, PGAP6, EEF1A1P11, ZC3H10, ERH, PTGER2, CCP110, DENND6A, COA1, PTGER4, MAN2A2, RALGPS1, TMEM179B, CDC42P6, LINC01355, MFN2, TYW5, PSMG2, H3P47, ZNF761, RPL10P16, EIF2S3B, SMARCC1, KLF2, MFSD8, EXOSC2, RABIF, PRDX3, GGPS1, SAP18, MBD3, AHCTF1P1, YWHAZP3, ENSG00000270066, MOGS, CARD8-AS1, KIAA2013, HPCAL1, SELENON, CDC42BPB, PCID2, TGFBRAP1, ENSG00000244313, THUMPD3, RAC1P2, ENSG00000283907, VDAC3, TBC1D25, PRRC2C, SLC16A5, ANKRA2, PTCD2, TRAM1. Genomic location of transcripts on hg38 reference genome can be determined via the Ensembl annotation guide v109. (https://ftp.ensembl.org/pub/release-109/gtf/homo_sapiens/Homo_sapiens.GRCh38.109.gtf.gz).

Consideration of genetic ancestry vs. race as a biological variable. Genetic tests to diagnose hypertrophic cardiac myopathy fail in African American patients, suggesting race specific diagnostic profiles need verification. Racial differences in epilepsy incidence over life and control of seizures are noted. The study tested diagnostic profiles across various racial categories. However, race is a cultural construct, accounting for social and environmental factors, as well as genetic influences on disease. To differentiate between these factors, the study focused on genetic ancestry as a factor. Using RNA-seq based inference the study identified proportion of European and African genetics in the participants to stratify the participants. RNA data was compared to self-reported data. The study determined whether correcting for genetic ancestry profiles when determining DEG profiles, or ancestry specific profiles was more accurate at predicting the occurrence of a seizure.

### Example 6. Development of Predictive Models

FIG. 12 shows a workflow to determine ability of RNA expression patterns for diagnosis prediction modeling. Briefly, blood samples from seizure patients and controls (PNES) are stratified based on VideoEEG reporting. All seizure types are grouped together (generalized and focal seizures). Samples are first analyzed at each time post seizure (6 h, 24 h 72 h and 7 day post ictus vs. baseline). Differentially expressed genes (DEGs) are identified using a linear model, correcting for batch effects using Partek or Limma. Age and sex proportions of samples used for modeling are approximately matched (and corrected for). RNA expression patterns correlating with seizure diagnosis are used as classifiers for modeling. Transcript isoform usage and exon expression are also assessed.

Prediction modeling. DEGs are used as classifiers for Support Vector Machine models (SVM). Data are partitioned using “full leave one out” method, and refined by testing gamma, shrinking variables. Models are further verified using an R implementation of SVM, to ensure reproducibility. Models with the highest normalized correct rate are tested further with the validation cohort of samples. AI/ML models (NVIDIA CUDA and R based packages) including random forest and neural network modeling, for multifactorial prediction models can also be used.

An integrated workbench is used for analysis. Partek is GUI based, and therefore simpler to use, while retaining a strong compliment of tools: including variable selection-ANOVA analysis of expression levels, shrinking centroids, with forward, backwards exhaustive and genetic algorithms, Data classification variables-k-nearest neighbors, Nearest centroid, Discriminant Analysis, Support vector machine and logistic regression, Distance measure candidates-Euclidean distance, average Euclidian, Pearson's and Spearman's absolute and dissimilarity models, Cross validation tests-1 level, 2-level nested, & bootstrap. Parametric approaches are used for data analysis because analysis of the data revealed a normal distribution.

Data can also be analyzyed using open source coding software packages in R. CombatSeq is used for removing batch effects, if not resolved in Partek. Gene expression and Transcript usage can be quantified using DESEQ2, EDGER, and LIMMA packages in R. The repeated measures effects of samples can be resolved by adding the participant ID as a dependent contrast variable, or using alternatives including Dream and rmRNAseq packages. An Ensembl reference annotation guide is used to create the pilot data. A custom gtf is used to help quantify orphan gene and novel RNAs. Prediction modeling can be performed in R and other statistical/mathematical packages including but not limited to matlab, statistica, spss, and python.

The contents of all references, patents, and published patent applications cited throughout this application, as well as the Figures and Tables, are incorporated herein by reference.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims.

What is claimed is:

1. A method for differentiating epileptic seizure from psychogenic non-epileptic seizure, the method comprising:
generating a transcriptome profile of a seizure suspect from a blood sample obtained from the seizure suspect after a suspected seizure event;
comparing the transcriptome profile of the seizure suspect to reference transcriptome profiles of seizure patients; and
determining whether the seizure suspect suffered seizure based on a result of the comparing step.

2. The method of claim 1, wherein the seizure suspect is deemed to have suffered a seizure if the transcriptome profile of the seizure suspect matches to a reference transcriptome profile of seizure patients.

3. The method of claim 1, further comprising the step of determining the seizure type, if the seizure suspect suffered a seizure.

4. The method of claim 3, wherein the step of determining the seizure type comprises the step of comparing the transcriptome profile of the seizure suspect to transcriptome profiles of seizure subtype.

5. The method of claim 1, wherein the transcriptome profile is a whole transcriptome profile generated from RNA extracted from whole blood.

6. The method of claim 1, further comprising the step of providing a recommendation for appropriate seizure treatment or seizure prevention, if the seizure suspect suffered a seizure.

7. The method of claim 1, further comprising the step of treating the seizure suspect with a seizure treatment, if the seizure suspect suffered a seizure.

8. The method of claim 7, wherein the seizure is epileptic seizure and wherein the seizure treatment comprises the step of administering to the seizure suspect, an effective amount of one or more drugs selected from the group consisting of brivaracetam, ezogabine, pregabalin, cannabidiol oral solution, felbamate, primidone, carbamazepine, fenfluramine, rufinamide, carbamazepine-XR, gabapentin, stiripentol, cenobamate, lacosamide, tiagabine hydrochloride, lamotrigine, clobazam, levetiracetam, topiramate, clonazepam, levetiracetam XR, topiramate XR, diazepam nasal, lorazepam, valproic acid, diazepam rectal, oxcarbazepine, vigabatrin, divalproex sodium-ER, phenobarbital, eslicarbazepine acetate, phenytoin and ethosuximide.

9. The method of claim 1, wherein the step of comparing the transcriptome profile of the seizure suspect to reference transcriptome profiles of seizure patients includes the consideration of biographical information of the seizure suspect.

10. The method of claim 1, wherein the step of comparing the transcriptome profile of the seizure suspect to reference transcriptome profiles of seizure patients includes the consideration of the sex and race of the seizure suspect.

11. A method of enhancing treatment of epileptic seizures, comprising the steps of:
obtaining a whole blood sample from a subject, wherein the subject is suspected to have experienced an epileptic seizure;
preparing an RNA library from the whole blood sample;
sequencing the RNA library;
determining differential expression of a plurality of RNA sequences comprised within the RNA library, wherein the plurality of RNA sequences comprises non-coding RNA (ncRNA);
creating a transcriptome profile based on the differential expression of the RNA sequences;
comparing the transcriptome profile to a reference transcriptome profile for epileptic seizure;
determining whether the subject suffered an epileptic seizure, based on the result of the comparing step; and
treating the subject with a therapy for epileptic seizure if the subject is deemed to have suffered an epileptic seizure in the determining step
determining when the patient suffered the seizure based on comparison to prediction model.

12. The method of claim 11, wherein the comprising step further comprises:
comparing the transcriptome profile to a reference transcriptome profile for non-epileptic seizure;

13. The method of claim 11, wherein the reference transcriptome profile for epileptic seizure is a reference transcriptome profile having the same sex and race characterization as the subject.

14. The method of claim 11, wherein the therapy comprises the step of administering to the subject, an effective amount of one or more drugs selected from the group consisting of brivaracetam, ezogabine, pregabalin, cannabidiol oral solution, felbamate, primidone, carbamazepine, fenfluramine, rufinamide, carbamazepine-XR, gabapentin, stiripentol, cenobamate, lacosamide, tiagabine hydrochloride, lamotrigine, clobazam, levetiracetam, topiramate, clonazepam, levetiracetam XR, topiramate XR, diazepam nasal, lorazepam, valproic acid, diazepam rectal, oxcarbazepine, vigabatrin, divalproex sodium-ER, phenobarbital, eslicarbazepine acetate, phenytoin and ethosuximide.

15. A method for treating epileptic seizures in a subject, comprising the steps of:
generating a transcriptome profile from a blood sample obtained from the subject;
confirming that the subject suffered an epileptic seizure by identifying epileptic seizure RNA signatures in the transcriptome profile of the subject, wherein the presence of epileptic seizure RNA signatures indicates that the subject suffered an epileptic seizure; and
treating the subject confirmed of epileptic seizure with a therapy for epileptic seizure.

16. The method of claim 15, wherein the therapy comprises the step of administering to the subject, an effective amount of one or more drugs selected from the group consisting of brivaracetam, ezogabine, pregabalin, cannabidiol oral solution, felbamate, primidone, carbamazepine, fenfluramine, rufinamide, carbamazepine-XR, gabapentin, stiripentol, cenobamate, lacosamide, tiagabine hydrochloride, lamotrigine, clobazam, levetiracetam, topiramate, clonazepam, levetiracetam XR, topiramate XR, diazepam nasal, lorazepam, valproic acid, diazepam rectal, oxcarbazepine, vigabatrin, divalproex sodium-ER, phenobarbital, eslicarbazepine acetate, phenytoin and ethosuximide.

17. A computer system for performing the analyzing and determining steps of method 1, comprising
a processor; and
a memory coupled to the processor; wherein the memory encodes one or more programs that cause the processor to perform the analyzing and determining steps of the method of claim 1.

18. The computer system of claim 17, wherein the one or more programs encoded in the memory cause the computer to access a seizure patient database to obtain reference transcriptome profiles of seizure patients.
